(19) United States
(12) Patent Application Publication
(10) Pub. No.: US 2019/0060311 A1
(43) Pub. Date: Feb. 28, 2019

(54) COMPOSITIONS AND METHODS FOR TREATMENT OF VITILIGO

(71) Applicant: ACLARIS THERAPEUTICS, INC., Wayne, PA (US)

(72) Inventors: Stuart D. SHANLER, Phoenixville, PA (US); Evan DICK, Phoenixville, PA (US); Neal Stuart WALKER, Phoenixville, PA (US); Christopher POWALA, Radnor, PA (US)

(21) Appl. No.: 16/110,017

(22) Filed: Aug. 23, 2018

Related U.S. Application Data
(60) Provisional application No. 62/549,831, filed on Aug. 24, 2017.

(51) Int. Cl. A61K 31/506; A61P 17/00; A61K 31/5377; A61K 31/496; A61K 31/519; A61K 31/517

(52) U.S. Cl. CPC A61K 31/506; A61P 17/00; A61K 31/517; A61K 31/496; A61K 31/519; A61K 31/5377

(57) ABSTRACT

Methods of treating vitiligo using JAK/STAT modulating compounds are provided herein. The JAK/STAT modulating compounds may be selected from 5-((2-((4-fluoro-3-methoxy-5-methylphenyl)amino)-5-methylpyrimidin-4-yl)amino)benzo[d] oxazol-2(3H)-one, disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, tofacitinib, ruxolitinib, baricitinib, lestaurtinib, decernotinib, TG101348, Janex 1, PF-956980, WHI-P154, ZM-39923, NSC114792, cerdulatinib, fedratinib, PF-06263276, CEP-33779, AZD-1480, SHR0302, oclacitinib, filgotinib, gandotinib, momelotinib, pacritinib, upadacitinib, peficitinib, PF-04965842, N-(3-acetamido-5-(quinaxalin-2-yl) phenyl) acrylamide, 1-[(2S,5R)-2-Methyl-5-(7H-pyrrolo[2,3-d]pyrimidin-4-ylamino)-1-piperidinyl]-2-propen-1-one malonate (PF-06651600), [(1S)-2,2-Difluorocyclopropyl][3-[2-[(1-methyl-1H-pyrazol-4-yl)amino]-4-pyrimidinyl]-3,8-diazabicyclo[3.2.1]oct-8-yl]-methanone tosylate, a derivative thereof, or a combination thereof. The JAK/STAT modulating compounds may be formulated for topical or oral delivery.

### CROSS-REFERENCE TO RELATED APPLICATIONS

The present application claims the benefit of U.S. Provisional Patent Application 62/549,831 filed Aug. 24, 2017, entitled, “COMPOSITIONS AND METHODS FOR THE TREATMENT OF VITILIGO,” the content of which is incorporated herein by reference in its entirety.

### SUMMARY

Embodiments herein are directed to a method of treating vitiligo in a subject in need thereof comprising administering to the subject a therapeutically effective amount of a JAK/STAT modulating compound. In some embodiments, the JAK/STAT modulating compound may be selected from 5-((2-((4-fluoro-3-methoxy-5-methylphenyl)amino)-5-methylpyrimidin-4-yl)amino)benzo[d]oxazol-2(3H)-one, disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, tofacitinib, ruxolitinib, baricitinib, lestaurtinib, decernotinib, TG101348, Janex 1, PF-956980, WHI-P154, ZM-39923, NSC114792, cerdulatinib, fedratinib, PF-06263276, CEP-33779, AZD-1480, SHR0302, oclacitinib, filgotinib, gandotinib, momelotinib, pacritinib, upadacitinib, peficitinib, PF-04965842, N-(3-acetamido-5-(quinaxalin-2-yl) phenyl) acrylamide, 1-[(2S,5R)-2-Methyl-5-(7H-pyrrolo[2,3-d]pyrimidin-4-ylamino)-1-piperidinyl]-2-propen-1-one malonate (PF-06651600), [(1S)-2,2-Difluorocyclopropyl][3-[2-[(1-methyl-1H-pyrazol-4-yl)amino]-4-pyrimidinyl]-3,8-diazabicyclo[3.2.1]oct-8-yl]-methanone tosylate, a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, and a combination thereof. In some embodiments, the JAK/STAT modulating compound is 5-((2-((4-fluoro-3-methoxy-5-methylphenyl)amino)-5-methylpyrimidin-4-yl)amino)benzo[d]oxazol-2(3H)-one or disodium (5-((2-((4-fluoro-3-methoxy-5-methylphenyl)amino)-5-methylpyrimidin-4-yl)amino)-2-oxobenzo[d]oxazol-3(2H)-yl)methyl phosphate. In some embodiments, the route of administration of the JAK/STAT modulating compound is selected from the group consisting of oral, topical, systemic, subcutaneous, intramuscular, intraperitoneal, transdermal, intravenous injection, and a combination thereof. In some embodiments, the JAK/STAT modulating compound is in an oral pharmaceutical composition, provided, however, that the JAK/STAT modulating compound is not tofacitinib or ruxolitinib. In some embodiments, the JAK/STAT modulating compound is topically administered at a dose from about 0.01% w/w to about 20% w/w. In some embodiments, the JAK/STAT modulating compound is orally administered at a dose from about 0.0001 μg/kg body weight to about 20,000 μg/kg body weight. In some embodiments, the JAK/STAT modulating compound is in a pharmaceutical composition further comprising a pharmaceutically acceptable excipient.

Some embodiments herein are directed to a method of treating vitiligo in a subject in need thereof comprising topically administering to the subject a JAK/STAT modulating compound selected from 5-((2-((4-fluoro-3-methoxy-5-methylphenyl)amino)-5-methylpyrimidin-4-yl)amino)benzo[d]oxazol-2(3H)-one, disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, tofacitinib, baricitinib, lestaurtinib, decernotinib, TG101348, Janex 1, PF-956980, WHI-P154, ZM-39923, NSC114792, cerdulatinib, fedratinib, PF-06263276, CEP-33779, AZD-1480, SHR0302, oclacitinib, filgotinib, gandotinib, momelotinib, pacritinib, upadacitinib, peficitinib, PF-04965842, N-(3-acetamido-5-(quinaxalin-2-yl) phenyl) acryl amide, 1-[(25,5R)-2-Methyl-5-(7H-pyrrolo[2,3-d]pyrimidin-4-ylamino)-1-piperidinyl]-2-propen-1-one malonate (PF-06651600), [(1S)-2,2-Difluorocyclopropyl][3-[2-[(1-methyl-1H-pyrazol-4-yl)amino]-4-pyrimidinyl]-3,8-diazabicyclo[3.2.1]oct-8-yl]-methanone tosylate, a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, and a combination thereof. In some embodiments, the JAK/STAT modulating compound is not ruxolitinib. In some embodiments, the JAK/STAT modulating compound is in a spray, extended release form, delayed release form, coated form, enteric coated form, elixir, suppository, liniment, lotion, shampoo, pastille, patch, pellet, pill, solution, powder, fluid emulsion, suspension, nanoparticle, nanoparticle suspension, nanocapsule, liposomes, nanosuspension, fluid suspension, semi-solid, ointment, paste, cream, gel, jelly, or foam.

In some embodiments, the vitiligo is segmental vitiligo, non-segmental vitiligo, focal vitiligo, generalized vitiligo, universal vitiligo, mucosal vitiligo or vitiligo with or without leukotricia (involvement of body hair).

Some embodiments are directed to a method of treating vitiligo in a subject in need thereof comprising orally administering to the subject a therapeutically effective amount of a JAK/STAT modulating compound. In some embodiments, the JAK/STAT modulating compound may be selected from 5-((2-((4-fluoro-3-methoxy-5-methylphenyl)amino)-5-methylpyrimidin-4-yl)amino)benzo[d]oxazol-2(3H)-one, disodium (5-((2-((4-fluoro-3-methoxy-5-methylphenyl)amino)-5-methylpyrimidin-4-yl)amino)-2-oxobenzo[d] oxazol-3(2H)-yl)methyl phosphate, baricitinib, lestaurtinib, decernotinib, TG101348, Janex 1, PF-956980, WHI-P154, ZM-39923, NSC114792, cerdulatinib, fedratinib, PF-06263276, CEP-33779, AZD-1480, SHR0302, oclacitinib, filgotinib, gandotinib, momelotinib, pacritinib, upadacitinib, peficitinib, PF-04965842, N-(3-acetamido-5-(quinaxalin-2-yl) phenyl) acrylamide, 1-[(2S,5R)-2-Methyl-5-(7H-pyrrolo[2,3-d]pyrimidin-4-ylamino)-1-piperidinyl]-2-propen-1-one malonate (PF-06651600), [(1S)-2,2-Difluorocyclopropyl][3-[2-[(1-methyl-1H-pyrazol-4-yl)amino]-4-pyrimidinyl]-3,8-diazabicyclo[3.2.1]oct-8-yl]-methanone tosylate, a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomers thereof, a racemate thereof, a mixtures of stereoisomer, or a combination thereof. In some embodiments, the JAK/STAT modulating compound is an oral pharmaceutical composition that does not include ruxolitinib or tofacitinib. In some embodiments, the JAK/STAT modulating compound is in a tablet, capsule, cachet, pellet, pill, powder, granules, or a combination thereof.

### DESCRIPTION OF THE FIGURES

FIG. 1 is directed to vitiligo scores for animals dosed with 0, 50 or 100 mg/kg 5-((2-((4-fluoro-3-methoxy-5-methylphenyl)amino)-5-methylpyrimidin-4-yl)amino)benzo[d]oxazol-2(3H)-one hydrochloride.

### DETAILED DESCRIPTION

Before the present compositions and methods are described, it is to be understood that this invention is not limited to the particular processes, formulations, compositions, or methodologies described, as these may vary. It is also to be understood that the terminology used in the description is for the purpose of describing the particular versions or embodiments only, and is not intended to limit the scope of embodiments herein which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of embodiments herein, the preferred methods, devices, and materials are now described. All publications mentioned herein are incorporated by reference in their entirety. Nothing herein is to be construed as an admission that embodiments herein is not entitled to antedate such disclosure by virtue of prior invention.

It must also be noted that as used herein and in the appended claims, the singular forms “a,” “an,” and “the” include plural reference unless the context clearly dictates otherwise. Thus, for example, reference to a “JAK inhibitor” is a reference to one or more JAK inhibitors and equivalents thereof known to those skilled in the art, and so forth.

As used herein, the term “about” means plus or minus 10% of the numerical value of the number with which it is being used. Therefore, about 50% means in the range of 45%-55%.

“Administering” when used in conjunction with a therapeutic means to administer a therapeutic directly into or onto a target tissue or to administer a therapeutic to a patient whereby the therapeutic positively impacts the tissue to which it is targeted. Thus, as used herein, the term “administering”, when used in conjunction with a JAK/STAT modulating compound, can include, but is not limited to, providing an JAK/STAT modulating compound into or onto the target tissue; providing an JAK/STAT modulating compound systemically to a patient by, e.g., intravenous injection whereby the therapeutic reaches the target tissue; providing an JAK/STAT modulating compound in the form of the encoding sequence thereof to the target tissue (e.g., by so-called gene-therapy techniques). “Administering” a composition may be accomplished by injection, topical administration, orally, or by either method in combination with other known techniques.

The term “subject” as used herein includes, but is not limited to, humans and non-human vertebrates such as wild, domestic, and farm animals. In certain embodiments, the subject described herein is an animal. In certain embodiments, the subject is a mammal. In certain embodiments, the subject is a human. In certain embodiments, the subject is a non-human animal. In certain embodiments, the subject is a non-human mammal. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

The term “improve” is used to convey that the compounds of embodiments herein change either the appearance, form, characteristics and/or the physical attributes of the tissue to which it is being provided, applied or administered. The change in form may be demonstrated by any of the following alone or in combination: enhanced appearance of the skin; at least partial remission, such as repigmentation of existing areas of depigmentation; uniformity of skin color; increased melanin production in white patches; repigmentation of skin; and/or reduced incidence of new areas of depigmentation.

The term “inhibit” includes the administration of a compound of embodiments herein to prevent the onset of the symptoms, alleviating the symptoms, or eliminating the disease, condition or disorder.

By “pharmaceutically acceptable”, it is meant the carrier, diluent or excipient must be compatible with the other ingredients of the formulation and not deleterious to the recipient thereof.

Unless otherwise indicated, the term “skin” means that outer integument or covering of the body, consisting of the dermis and the epidermis and resting upon subcutaneous tissue.

As used herein, the term “therapeutic” means an agent utilized to treat, combat, inhibit, ameliorate, prevent or improve an unwanted condition or disease of a patient. In part, embodiments of embodiments herein are directed to the treatment of vitiligo.

A “therapeutically effective amount” or “effective amount” of a composition is a predetermined amount calculated to achieve the desired effect, i.e., to induce a favorable immunological response such as, e.g., a decrease in the number of T-cells in the skin (a marker of disease activity in vitiligo) or a decrease in the C-X-C motif chemokine ligand 9 (CXCL9) as measured by skin biopsy or suction blister fluid sampling, or to inhibit, block, or reverse depigmentation of skin. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, concomitant therapies and the condition being treated. The compounds are effective over a wide dosage range and, for example, dosages per day will normally fall within the range of from about 0.0001 μg/kg to about 20.000 μg/kg, However, it will be understood that the effective amount administered will be determined by the physician in the light of the relevant circumstances including the condition to be treated, the choice of compound to be administered, and the chosen route of administration, and therefore the above dosage ranges are not intended to limit the scope of embodiments herein in any way. A therapeutically effective amount of a compound of this disclosure is typically an amount such that when it is administered in a physiologically tolerable excipient composition, it is sufficient to achieve an effective systemic concentration or local concentration in the tissue.

The terms “treat,” “treated,” or “treating” as used herein refers to both therapeutic treatment and prophylactic or preventative measures, wherein the object is to inhibit, prevent or slow down (lessen) an undesired physiological condition, disorder or disease, or to improve, inhibit, or otherwise obtain beneficial or desired clinical results. For the purposes of this invention, beneficial or desired clinical results include, but are not limited to, improvement or alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

As used herein, a “JAK/STAT modulating compound” refers to a compound that interacts with a JAK1, JAK2, JAK3, TYK2, Stat 1, Stat 2, Stat 3, Stat 4, Stat 5a, Stat 5b, or Stat 6 gene or a JAK1, JAK2, JAK3, TYK2, Stat 1, Stat 2, Stat 3, Stat 4, Stat 5a, Stat 5b, or Stat 6 protein or polypeptide and modulates its activity and/or its expression. The compound can either increase the activity or expression of a protein encoded by a JAK 1, JAK 2, JAK3, TYK2, Stat 1, Stat 2, Stat 3, Stat 4, Stat 5a, Stat 5b, or Stat 6 gene. Conversely, the compound can decrease the activity or expression of a protein encoded by a JAK1, JAK2, JAK3, TYK2, Stat 1, Stat 2, Stat 3, Stat 4, Stat 5a, Stat 5b, or Stat 6 gene. The compound can be a JAK1, JAK2, JAK3, TYK2, Stat 1, Stat 2, Stat 3, Stat 4, Stat 5a, Stat 5b, or Stat 6 agonist or a JAK1, JAK2, JAK3, TYK2, Stat 1, Stat 2, Stat 3, Stat 4, Stat 5a, Stat 5b, or Stat 6 antagonist (e.g., a JAK1 inhibitor, a JAK2 inhibitor a JAK3 inhibitor, a TYK2 inhibitor, a Stat1 inhibitor, a Stat2 inhibitor, a Stat 3 inhibitor, a Stat 4 inhibitor, a Stat 5a inhibitor, a Stat 5b inhibitor, or a Stat 6 inhibitor). Some non-limiting examples of JAK1, JAK2, JAK3, TYK2, Stat 1, Stat 2, Stat 3, Stat 4, Stat 5a, Stat 5b, or Stat 6 modulating compounds include peptides (such as peptide fragments comprising a polypeptide encoded by a JAK1, JAK2, JAK3, TYK2, Stat 1, Stat 2, Stat 3, Stat 4, Stat 5a, Stat 5b, or Stat 6 gene, or antibodies or fragments thereof), small molecules, and nucleic acids (such as siRNA or antisense RNA specific for a nucleic acid comprising a JAK1, JAK2, JAK3, TYK2, Stat 1, Stat 2, Stat 3, Stat 4, Stat 5a, Stat 5b, or Stat 6 gene). Agonists of a JAK1, JAK2, JAK3, TYK2, Stat 1, Stat 2, Stat 3, Stat 4, Stat 5a, Stat 5b, or Stat 6 protein can be molecules which, when bound to a JAK1, JAK2, JAK3, TYK2, Stat 1, Stat 2, Stat 3, Stat 4, Stat 5a, Stat 5b, or Stat 6 protein, increase or prolong the activity of the JAK1, JAK2, JAK3, TYK2, Stat 1, Stat 2, Stat 3, Stat 4, Stat 5a, Stat 5b, or Stat 6 protein. JAK1, JAK2, JAK3, TYK2, Stat 1, Stat 2, Stat 3, Stat 4, Stat 5a, Stat 5b, or Stat 6 agonists include, but are not limited to, proteins, nucleic acids, small molecules, or any other molecule which activates a JAK1, JAK2, JAK3, TYK2, Stat 1, Stat 2, Stat 3, Stat 4, Stat 5a, Stat 5b, or Stat 6 protein. Antagonists of a JAK1, JAK2, JAK3, TYK2, Stat 1, Stat 2, Stat 3, Stat 4, Stat 5a, Stat 5b, or Stat 6 protein can be molecules which, when bound to a JAK1, JAK2, JAK3, TYK2, Stat 1, Stat 2, Stat 3, Stat 4, Stat 5a, Stat 5b, or Stat 6 protein decrease the amount or the duration of the activity of the JAK1, JAK2, JAK3, TYK2, Stat 1, Stat 2, Stat 3, Stat 4, Stat 5a, Stat 5b, or Stat 6 protein. Antagonists include proteins, nucleic acids, antibodies, small molecules, or any other molecule which decrease the activity of a JAK1, JAK2, JAK3, TYK2, Stat 1, Stat 2, Stat 3, Stat 4, Stat 5a, Stat 5b, or Stat 6 protein.

The term “modulating,” as it appears herein, refers to a change in the activity or expression of a gene or protein of JAK1, JAK2, JAK3, TYK2, Stat 1, Stat 2, Stat 3, Stat 4, Stat 5a, Stat 5b, or Stat 6. For example, modulation can cause an increase or a decrease in protein activity, binding characteristics, or any other biological, functional, or immunological properties of a JAK1, JAK2, JAK3, TYK2, Stat 1, Stat 2, Stat 3, Stat 4, Stat 5a, Stat 5b, or Stat 6 protein.

Vitiligo is a common, disfiguring autoimmune disease caused by the destruction of melanocytes. Vitiligo is a condition that causes depigmentation of skin, typically in sections or patches, and affects about 1-2% of the world population. Vitiligo occurs when there is an absence of functional melanocytes (melanin-producing cells) in the skin. Vitiligo also can affect the mucous membranes and the eye. People with vitiligo may be at increased risk of social or psychological distress, sunburn, skin cancer, eye problems, such as inflammation of the iris (iritis), and hearing loss. There may be a genetic predisposition to vitiligo in some cases. The average age at vitiligo onset is about 20 years, with onset most commonly observed between the ages of 10 and 30.

Vitiligo occurs most often on the face and extremities—typically the hands and wrists. Depigmentation also can occur around the mouth, eyes, nostrils, genitalia, and umbilicus. Depigmented patches are flat areas of normal-feeling skin, and may have a hyperpigmented edge. The edges typically are well-defined but irregular. In trichrome vitiligo, there is an intermediate zone of hypochromia between the achromic center and peripheral unaffected skin.

There are several clinical classifications of vitiligo. Segmental vitiligo presents as one or more macules in a dermatomal or quasidermatomal pattern, and occurs most commonly in children. All other types of vitiligo are classified as non-segmental vitiligo, which is most common. Focal vitiligo is characterized by depigmentation in one area, or macule, such as the trigeminal nerve distribution. Other forms of non-segmental vitiligo often produce symmetric patches, sometimes covering large areas. Mucosal vitiligo affects only mucosal membranes. Generalized vitiligo may be acrofacial, in which depigmentation occurs on the distal fingers and periorificial areas, or vulgaris, which is characterized by widely distributed, scattered patches. Universal vitiligo manifests as complete or nearly complete depigmentation, and frequently is associated with multiple endocrinopathy syndrome. The exact cause of melanocyte loss in vitiligo remains debatable, but recent observations have pointed to a role for cellular immunity in the pathogenesis of vitiligo (see, for example, Wang et al. (2011) Th17 Cells and Activated Dendritic Cells Are Increased in Vitiligo Lesions. PLoS ONE 6(4): e18907). Despite some advances in elucidating the origins of the disorder, current therapies, such as topical corticosteroids, topical immunomodulators and psoralen phototherapy have serious side effects and limited therapeutic utility. Vitiligo is a disfiguring disease for which current therapies have proven unsatisfactory.

Because the interplay between the potential pathophysiologic mechanisms involved in the development of vitiligo (e.g., the role of intrinsic melanocyte abnormalities, innate immune activation, adaptive immunity) is complex and may be difficult to define using only human individuals, numerous preclinical animal models have been developed that provide an opportunity to study the pathogenesis of vitiligo and to suggest potentially successful approaches to the treatment of humans with or predisposed to develop the condition. Additionally, because of distinctions between human melanocytes (such as, e.g., at different anatomical locations), and interspecies differences in melanocytes, no single model may be ideal. However, both spontaneous and induced models of vitiligo have been identified and developed, each with distinct advantages, that enable investigation of immune events during disease initiation and progression and that offer insight into potentially valuable ways to either prevent disease development, modify its course or phenotypic expression, induce or maintain a partial or complete disease remission, or potentially cure forms of the disease. Importantly, findings of these basic and translational research studies using a variety of animal models of disease, have identified important pathophysiological mechanisms in the development and maintenance of vitiligo. For example, studies in mouse models of vitiligo have demonstrated that the activity of the IFN-γ-chemokine signaling axis is important in vitiligo and that blockade of this pathway in a mouse model could disrupt T-cell recruitment to the skin, resulting in a subsequent decrease of melanocyte destruction and pigment loss in the animal. In fact, similar to the mouse model, blockade of the IFN-γ pathway has been demonstrated to successfully treat vitiligo in humans as well.

The Janus kinase (JAK) family comprises a family of cytoplasmic, non-receptor tyrosine kinases, that can transduce cytokine-mediated signals to activate the STAT transcription factors. The JAK signal transducer and activator (STAT) of transcription (JAK/STAT) pathway can be utilized by cytokines including at least interleukins (ILs), interferons (IFNs), and other molecules that can transmit signals from the cell membrane to the nucleus. Upon engagement of extracellular ligands, intracellular JAK proteins, which can associate with type I/II cytokine receptors, may become activated and phosphorylate STAT proteins, which can dimerize and may translocate into the nucleus to directly or indirectly regulate gene expression. The JAK family of kinases may include at least JAK1, JAK2, JAK3, and tyrosine kinase 2 (Tyk2). Individual JAKs can associate with different receptors, but because at least 4 JAKs exist, each member can be used by multiple different receptors. Growth factors or cytokine receptors that recruit JAK kinases include the interferon receptors, interleukin receptors (receptors for the cytokines IL-2 to IL-7, IL9 to IL-13, IL-15, and IL-23), various hormone receptors (erythropoietin receptor, the thrombopoietin receptor, the leptin receptor, the insulin receptor, the prolactin receptor, the granulocyte colony-stimulating factor, the growth hormone receptor, receptor protein tyrosine kinases, and receptors for other growth factors such as leukemia inhibitory factor, oncostatin M, IFNα/β/γ, ciliary neutotrophic factor, and cardiotrophin-1.

Phosphorylated receptors serve as docking sites for other SH-2 domain containing signaling molecules that interact with JAKs such as the STAT family of transcription factors, Src family of kinases, MAP kinases, PI3 kinase and protein tyrosine phosphatases. The family of latent cytoplasmic transcription factors, STATs, is the most well characterized downstream substrates for JAKs. The STAT proteins bind to phosphorylated cytokine receptors through their SH2 domains to become phosphorylated by JAKs, which leads to their dimerization and release and eventual translocation to the nucleus where they activate gene transcription. The various members of STAT which have been identified thus far, are STAT1, STAT2, STAT3, STAT4, STATS (including STAT5a and STAT5b) and STATE.

JAK1 and JAK2 can be involved in IFN-γ signal transduction following its binding to the receptor, and are thus downstream signaling mediators that could be appropriate targets for vitiligo therapy. Additionally, inhibitors of IL-15 such as JAK1 & JAK3 and inhibitors/downregulators of the recently elucidated relevant JAK/STAT pathways can be targets for inhibiting the immune mediated inflammatory responses driving immune mediated diseases such as, inter alia, alopecia areata and vitiligo, as they can be associated with psoriasis, atopic dermatitis, the autoimmune arthritides, and certain cancers and myeloproliferative diseases.

Embodiments herein are directed to methods of treating vitiligo in a subject in need thereof, the method comprising administering to the subject a JAK/STAT modulating compound. In some embodiments, a method of treating vitiligo in a subject in need thereof may comprise administering to the subject a therapeutically effective amount of a JAK/STAT modulating compound. In some embodiments, said vitiligo is segmental vitiligo including unisegmental, bisegmental or multisegmental vitiligo, non-segmental vitiligo including acral, facial, or acrofacial vitiligo, centrofacial vitiligo, mucosal vitiligo, confetti vitiligo, trichrome vitiligo, marginal inflammatory vitiligo, quadrichrome vitiligo, blue vitiligo, Koebner phenomenon vitiligo, vulgaris vitiligo, generalized vitiligo, universal vitiligo, mixed vitiligo (nonsegmental associated with segmental vitiligo), focal vitiligo, solitary mucosal vitiligo or vitiligo with or without leukotricia (involvement of body hair) or any type of vitiligo set forth in Table 1 below:

TABLE 1
Classification of vitiligo.

| NOMEN- | | |
|---|---|---|
| CLATURE | SUBSET | NOTES |
| Non-segmental | Acrofacial | Usually limited to face, head, hands, |
| vitiligo | | and feet |
| | Generalized | Symmetrical macules, mainly hands, |
| | | fingers, face, and trauma-exposed |
| | | areas |
| | Mucosal (at | Involvement of the oral and/or |
| | least two sites | genital mucosae with other sites of |
| | involved) | skin involvement |
| | Universal | Depigmentation affects 80%-90% of |
| | | body surface. |
| Segmental | Unisegmental | One or more depigmented macules |
| vitiligo | | distributed on one side of the body |
| | Bisegmental | Two segmental lesions distributed |
| | | either unilaterally or bilaterally |
| | Plurisegmental | Multiple segmental lesions |
| | | distributed either unilaterally or |
| | | bi-laterally |
| Mixed vitiligo | Occurence of | SV followed by NSV with a delay |
| | SV and NSV | of at least 6 months. At least 20% of |
| | | a dermatomal segment affected by |
| | | SV. |
| Unclassified | Focal vitiligo | Isolated macules that do not have a |
| vitiligo | | segmental distribution. No evolution |
| | | into NSV after at least 2 years |
| | Mucosal | Exclusive involvement of the oral or |
| | vitiligo (only | genital mucosae |
| | one site | |
| | involved) | |

In some embodiments, the compounds can be used for the treatment of vitiligo (e.g. localized vitiligo, focal vitiligo, generalized vitiligo, segmental vitiligo, acral vitiligo, facial vitiligo, acrofacial vitiligo, mucosal vitiligo, confetti vitiligo, trichrome vitiligo, marginal inflammatory vitiligo, quadrichrome vitiligo, blue vitiligo, Koebner phenomenon vitiligo, vulgaris vitiligo, mixed acrofacial and vulgaris vitiligo, or universal vitiligo) alone or in combination with topical or systemic corticosteroids, topical calcineurin inhibitors, such as tacrolimus or topical pimecrolimus, phototherapy such as with heliotherapy, natural or artificial sunlight therapy, ultraviolet light therapy with UVB, narrow-band UVB, UVA, oral or topical photochemotherapy using (e.g. 8-methoxypsoralen (8-MOP)), or psoralen plus ultraviolet A (PUVA), calcipotriene or other topical vitamin D analogs, laser therapy, such as (e.g.) with the excimer laser (308 nm) or helium-neon (HeNe) laser, systemic immunosuppressive agents (e.g. cyclosporine A, azathioprene, cyclophosphamide), topical or oral khellin with or without phototherapy with natural sunlight or UVA, topical or oral L-phenylalanine with or without phototherapy with natural sunlight or UVA or UVB, topical and oral antioxidants (e.g. vitamin E, vitamin C, alphalipoic acid, ginkgo biloba, topical catalase, superoxide dismutase, polypodium leucotomos), surgical treatments such as skin minigrafting, blister grafting, punch grafting, curettage grafting, split-thickness skin grafting, transplantation of autologous melanocyte suspension, camouflage such as with make-up, tattoos (permanent dermal micropigmentation) or self-tanning agents such as dihydroxyacetone (DHA) and such, other systemic agents such as tumor necrosis factor alpha (TNFα) inhibitors, antibiotics with anti-inflammatory or immunomodulatory properties such as the tetracyclines, or other therapies known to have beneficial effects in the condition.

In some embodiments herein, any known inhibitor of a protein tyrosine kinase (PTK) involved in cytokine signaling, such as JAK/STAT proteins JAK1, JAK2, JAK3, TYK2, Stat 1, Stat 2, Stat 3, Stat 4, Stat 5a, Stat 5b, or Stat 6 can be used for the treatment of vitiligo. In some embodiments, the JAK/STAT modulating compound is an inhibitor of a protein tyrosine kinase (PTK) involved in cytokine signaling. In some embodiments, the inhibitor is a JAK/Stat inhibitor.

In some embodiments, the JAK/STAT modulating compound is 5-((2-((4-fluoro-3-methoxy-5-methylphenyl)amino)-5-methylpyrimidin-4-yl)amino)benzo[d]oxazol-2(3H)-one, disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, tofacitinib, ruxolitinib, baricitinib, lestaurtinib, decernotinib, TG101348, Janex 1, PF-956980, WHI-P154, ZM-39923, NSC114792, cerdulatinib, fedratinib, PF-06263276, CEP-33779, AZD-1480, SHR0302, oclacitinib, filgotinib, gandotinib, momelotinib, pacritinib, upadacitinib, peficitinib, PF-04965842, N-(3-acetamido-5-(quinaxalin-2-yl) phenyl) acrylamide, 1-[(2S,5R)-2-Methyl-5-(7H-pyrrolo[2,3-d]pyrimidin-4-ylamino)-1-piperidinyl]-2-propen-1-one malonate (PF-06651600), [(1S)-2,2-Difluorocyclopropyl][3-[2-[(1-methyl-1H-pyrazol-4-yl)amino]-4-pyrimidinyl]-3,8-diazabicyclo[3.2.1]oct-8-yl]-methanone tosylate, or a combination thereof. In some embodiments, the JAK/STAT modulating compound is 5-((2-((4-fluoro-3-methoxy-5-methylphenyl)amino)-5-methylpyrimidin-4-yl)amino)benzo[d]oxazol-2(3H)-one. In some embodiments, the JAK/STAT modulating compound is disodium (5-((2-((4-fluoro-3-methoxy-5-methylphenyl)amino)-5-methylpyrimidin-4-yl)amino)-2-oxobenzo[d]oxazol-3(2H)-yl)methyl phosphate. In some embodiments, the JAK/STAT modulating compound is not ruxolitinib. In some embodiments, the JAK/STAT modulating compound is not tofacitinib. In some embodiments, the JAK/STAT modulating compound is in a pharmaceutical composition that does not include ruxolitinib. In some embodiments, the JAK/STAT modulating compound is in a pharmaceutical composition that does not include tofacitinib.

In some embodiments, a method of treating vitiligo in a subject in need thereof, the method comprising topically administering a therapeutically effect amount of a JAK/STAT modulating compound selected from 5-((2-((4-fluoro-3-methoxy-5-methylphenyl)amino)-5-methylpyrimidin-4-yl)amino)benzo[d]oxazol-2(3H)-one, disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, tofacitinib, ruxolitinib, baricitinib, lestaurtinib, decernotinib, TG101348, Janex 1, PF-956980, WHI-P154, ZM-39923, NSC114792, cerdulatinib, fedratinib, PF-06263276, CEP-33779, AZD-1480, SHR0302, oclacitinib, filgotinib, gandotinib, momelotinib, pacritinib, upadacitinib, peficitinib, PF-04965842, N-(3-acetamido-5-(quinaxalin-2-yl) phenyl) acrylamide, 1-[(2S,5R)-2-Methyl-5-(7H-pyrrolo[2,3-d]pyrimidin-4-ylamino)-1-piperidinyl]-2-propen-1-one malonate (PF-06651600), [(1S)-2,2-Difluorocyclopropyl][3-[2-[(1-methyl-1H-pyrazol-4-yl)amino]-4-pyrimidinyl]-3,8-diazabicyclo[3.2.1]oct-8-yl]-methanone tosylate, or combinations thereof. In some embodiments, the JAK/STAT modulating compound is not ruxolitinib. In some embodiments, the JAK/STAT modulating compound is in a pharmaceutical composition that does not include ruxolitinib.

In some embodiments, a method of treating vitiligo in a subject in need thereof, the method comprising topically administering a therapeutically effect amount of at least one JAK/STAT modulating compound selected from 5-((2-((4-fluoro-3-methoxy-5-methylphenyl)amino)-5-methylpyrimidin-4-yl)amino)benzo[d]oxazol-2(3H)-one, disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, tofacitinib, baricitinib, lestaurtinib, decernotinib, TG101348, Janex 1, PF-956980, WHI-P154, ZM-39923, NSC114792, cerdulatinib, fedratinib, PF-06263276, CEP-33779, AZD-1480, SHR0302, oclacitinib, filgotinib, gandotinib, momelotinib, pacritinib, upadacitinib, peficitinib, PF-04965842, N-(3-acetamido-5-(quinaxalin-2-yl) phenyl) acrylamide, 1-[(2S,5R)-2-Methyl-5-(7H-pyrrolo[2,3-d]pyrimidin-4-ylamino)-1-piperidinyl]-2-propen-1-one malonate (PF-06651600), [(1S)-2,2-Difluorocyclopropyl][342-[(1-methyl-1H-pyrazol-4-yl)amino]-4-pyrimidinyl]-3,8-diazabicyclo[3.2.1]oct-8-yl]-methanone tosylate, or combinations thereof. In some embodiments, the method of treating vitiligo in a subject in need thereof does not include topically administering ruxolitinib.

In some embodiments, a method of treating vitiligo in a subject in need thereof, the method comprising orally administering an effective amount of at least one JAK/STAT modulating compound selected from 5-((2-((4-fluoro-3-methoxy-5-methylphenyl)amino)-5-methylpyrimidin-4-yl)amino)benzo[d]oxazol-2(3H)-one, disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, baricitinib, lestaurtinib, decernotinib, TG101348, Janex 1, PF-956980, WHI-P154, ZM-39923, NSC114792, cerdulatinib, fedratinib, PF-06263276, CEP-33779, AZD-1480, SHR0302, oclacitinib, filgotinib, gandotinib, momelotinib, pacritinib, upadacitinib, peficitinib, PF-04965842, N-(3-acetamido-5-(quinaxalin-2-yl) phenyl) acrylamide, 1-[(2S,5R)-2-Methyl-5-(7H-pyrrolo[2,3-d]pyrimidin-4-ylamino)-1-piperidinyl]-2-propen-1-one malonate (PF-06651600), [(1S)-2,2-Difluorocyclopropyl][3-[2-[(1-methyl-1H-pyrazol-4-yl)amino]-4-pyrimidinyl]-3,8-diazabicyclo[3.2.1]oct-8-yl]-methanone tosylate, or combinations thereof. In some embodiments, the method does not include ruxolitinib or tofacitinib. Some embodiments are directed to a method of treating vitiligo comprising administering an oral pharmaceutical composition comprising an effective amount of 5-((2-((4-fluoro-3-methoxy-5-methylphenyl)amino)-5-methylpyrimidin-4-yl)amino)benzo[d]oxazol-2(3H)-one, disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, baricitinib, lestaurtinib, decernotinib, TG101348, Janex 1, PF-956980, WHI-P154, ZM-39923, NSC114792, cerdulatinib, fedratinib, PF-06263276, CEP-33779, AZD-1480, SHR0302, oclacitinib, filgotinib, gandotinib, momelotinib, pacritinib, upadacitinib, peficitinib, PF-04965842, N-(3-acetamido-5-(quinaxalin-2-yl) phenyl) acrylamide, or combinations thereof, wherein the composition does not include tofacitinib, ruxolitinib, 1-[(2S,5R)-2-Methyl-5-(7H-pyrrolo[2,3-d]pyrimidin-4-ylamino)-1-piperidinyl]-2-propen-1-one malonate (PF-06651600), [(1S)-2,2-Difluorocyclopropyl][3-[2-[(1-methyl-1H-pyrazol-4-yl)amino]-4-pyrimidinyl]-3,8-diazabicyclo[3.2.1]oct-8-yl]-methanone tosylate, or a combination thereof.

In some embodiments, the JAK/STAT modulating compound is 5-((2-((4-fluoro-3-methoxy-5-methylphenyl)amino)-5-methylpyrimidin-4-yl)amino)benzo[d]oxazol-2(3H)-one having the following structural formula

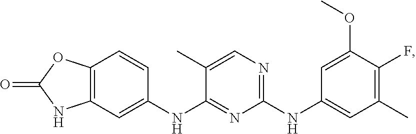 [SMILES: COc1cc(Nc2ncc(C)c(Nc3ccc4oc(=O)[nH]c4c3)n2)cc(C)c1F]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof. In some embodiments, the 5-((2-((4-fluoro-3-methoxy-5-methylphenyl)amino)-5-methylpyrimidin-4-yl)amino)benzo[d]oxazol-2(3H)-one is in a besylate salt form having the following structural formula:

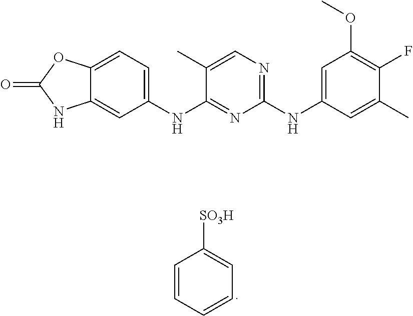 [SMILES: COc1cc(Nc2ncc(C)c(Nc3ccc4oc(=O)[nH]c4c3)n2)cc(C)c1F ; O=S(=O)(O)c1ccccc1]

In some embodiments, the 5-((2-((4-fluoro-3-methoxy-5-methylphenyl)amino)-5-methylpyrimidin-4-yl)amino)benzo[d]oxazol-2(3H)-one is in a trifluoroacetate salt form having the following structural formula:

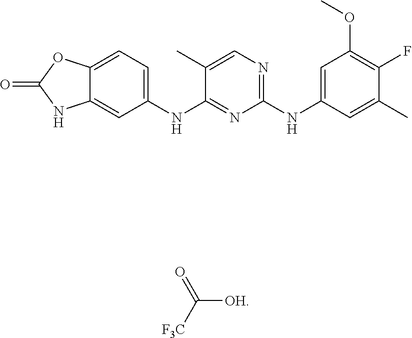 [SMILES: COc1cc(Nc2ncc(C)c(Nc3ccc4oc(=O)[nH]c4c3)n2)cc(C)c1F ; O=C(O)C(F)(F)F]

In some embodiments, the 5-((2-((4-fluoro-3-methoxy-5-methylphenyl)amino)-5-methylpyrimidin-4-yl)amino)benzo[d]oxazol-2(3H)-one is a hydrochloride salt form having the following structural formula:

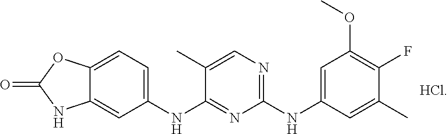 [SMILES: COc1cc(Nc2ncc(C)c(Nc3ccc4oc(=O)[nH]c4c3)n2)cc(C)c1F]

In some embodiments, the JAK/STAT modulating compound is disodium (5-((2-((4-fluoro-3-methoxy-5-methylphenyl)amino)-5-methylpyrimidin-4-yl)amino)-2-oxobenzo[d]oxazol-3(2H)-yl)methyl phosphate having the following structural formula

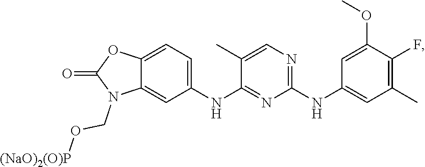

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is tofacitinib (3-{(3R,4R)-4-methyl-3-[methyl-(7H-pyrrolo[2,3-d]pyrimidin-4-yl)-amino]-piperidin-1-yl}-3-oxo-propionitrile; CAS No. 477600-75-2) having the chemical formula C₁₆H₂ON₆O and the following structural formula

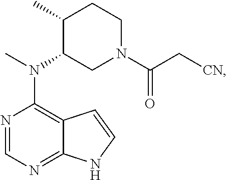 [SMILES: C[C@@H]1CCN(C(=O)CC#N)C[C@@H]1N(C)c1ncnc2[nH]ccc12]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is ruxolitinib ((R)-3-(4-(7H-pyrrolo[2,3-d]pyrimidin-4-yl)-1H-pyrazol-1-yl)-3-cyclopentylpropanenitrile; CAS No. 941678-49-5) having the following structural formula

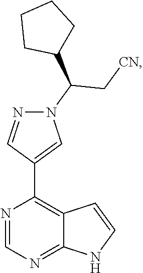 [SMILES: N#CC[C@H](C1CCCC1)n1cc(-c2ncnc3[nH]ccc23)cn1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is baricitinib (2-(3-(4-(7H-pyrrolo[2,3-d]pyrimidin-4-yl)-1H-pyrazol-1-yl)-1-(ethyl sulfonyl)azetidin-3-yl)acetonitrile; CAS No. 1187594-09-7) having the following structural formula,

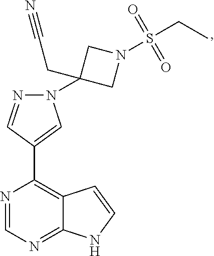 [SMILES: CCS(=O)(=O)N1CC(CC#N)(n2cc(-c3ncnc4[nH]ccc34)cn2)C1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is lestaurtinib ((5S,6S,8R)-6-hydroxy-6-(hydroxymethyl)-5-methyl-5,6,7, 8,14,15-hexahydro-13H-16-oxa-4b,8a,14-triaza-5,8-methanodibenzo[b,h]cycloocta[jkl]cyclopenta[e]-as-indacen-13-one; CAS No. 111358-88-4) having the following structural formula

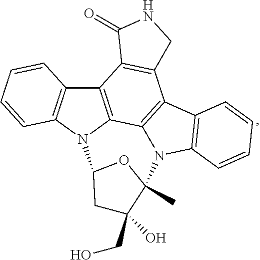 [SMILES: C[C@]12O[C@H](C[C@]1(O)CO)n1c3ccccc3c3c4c(c5c6ccccc6n2c5c31)CNC4=O]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is TG101348 (N-(tert-butyl)-3-((5-methyl-2-((4-(2-(pyrrolidin-1-yl)ethoxy)phenyl)amino)pyrimidin-4-yl)amino)benzenesulfonamide; CAS No. 936091-26-8) having the following structural formula

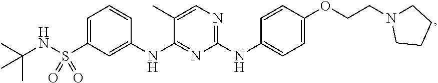 [SMILES: Cc1cnc(Nc2ccc(OCCN3CCCC3)cc2)nc1Nc1cccc(S(=O)(=O)NC(C)(C)C)c1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is decernotinib (2-((2-(1H-pyrrolo[2,3-b]pyridin-3-yl)pyrimidin-4-yl)amino)-2-methyl -N-(2,2,2-trifluoroethyl)butanamide; CAS No. 944842-54-0) having the following structural formula

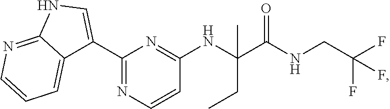 [SMILES: CCC(C)(Nc1ccnc(-c2c[nH]c3ncccc23)n1)C(=O)NCC(F)(F)F]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is Janex 1 (4-((6,7-dimethoxyquinazolin-4-yl)amino)phenol; CAS No. 202474-60-3) having the following structural formula

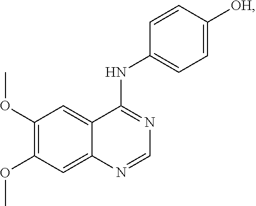 [SMILES: COc1cc2ncnc(Nc3ccc(O)cc3)c2cc1OC]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is PF-956980 (((3R,4R)-4-methyl-3-(methyl(7H-pyrrolo[2,3-d]pyrimidin-4-yl)amino)piperidin-1-yl)(pyrrolidin-1-yl)methanone) having the following structural formula

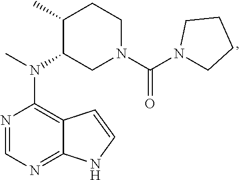 [SMILES: C[C@@H]1CCN(C(=O)N2CCCC2)C[C@@H]1N(C)c1ncnc2[nH]ccc12]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is WHI-P154 (2-bromo-4-((6,7-dimethoxyquinazolin-4-yl)amino)phenol; CAS No. 211555-04-3) having the following structural formula

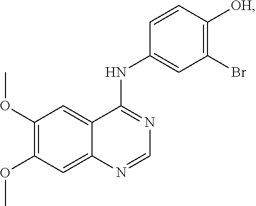 [SMILES: COc1cc2ncnc(Nc3ccc(O)c(Br)c3)c2cc1OC]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is ZM-39923 (3-(benzyl(isopropyl)amino)-1-(naphthalen-2-yl)propan-1-one) having the following structural formula

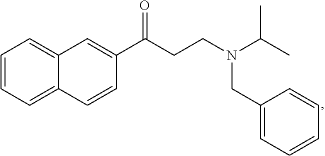 [SMILES: CC(C)N(CCC(=O)c1ccc2ccccc2c1)Cc1ccccc1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is NSC114792 (10,13-dimethyl-17-(2-(6-thioxo-3,6-dihydro-9H-purin-9-yl)acetyl)-1,2,6,7,8,9,10,11,12,13,14,15,16,17-tetradecahydro-3H-cyclopenta[a]phenanthren-3-one) having the following structural formula

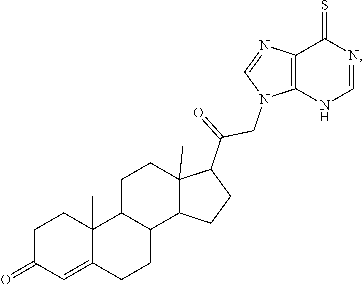 [SMILES: CC12CCC(=O)C=C1CCC1C2CCC2(C)C(C(=O)Cn3cnc4c(=S)nc[nH]c43)CCC12]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is SHR0302 (N-(3-methoxy-1,2,4-thiadiazol -5-yl)-5-(methyl(7H-pyrrolo[2,3-d]pyrimidin-4-yl)amino)hexahydrocyclopenta[c]pyrrole-2(1H)-carboxamide) having the following structural formula

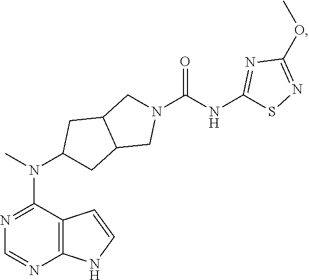 [SMILES: COc1nsc(NC(=O)N2CC3CC(N(C)c4ncnc5[nH]ccc45)CC3C2)n1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is AZD-1480 ((S)-5-chloro-N2-(1-(5-fluoropyrimidin-2-yl)ethyl)-N4-(5-methyl-1H-pyrazol-3-yl)pyrimidine-2,4-diamine) having the following structural formula

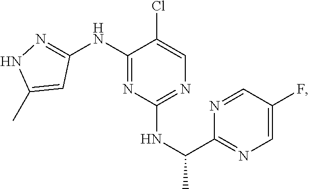 [SMILES: Cc1cc(Nc2nc(N[C@@H](C)c3ncc(F)cn3)ncc2Cl)n[nH]1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is CEP-33779 (N-(3-(4-methylpiperazin-1-yl)phenyl)-8-(4-(methyl sulfonyl)phenyl)-[1,2,4]triazolo[1,5-a]pyridin-2-amine) having the following structural formula

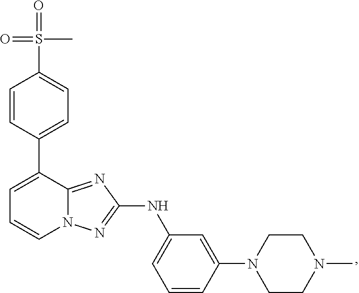 [SMILES: CN1CCN(c2cccc(Nc3nc4c(-c5ccc(S(C)(=O)=O)cc5)cccn4n3)c2)CC1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is PF-06263276 ((2-(6-(2-ethyl-5-fluoro-4-hydroxyphenyl)-1H-indazol -3-yl)-3,4,6, 7-tetrahydro-5H-imidazo[4,5-c]pyridin-5-yl)(5-(piperidin-1-yl)pyrazin-2-yl)methanone) having the following structural formula

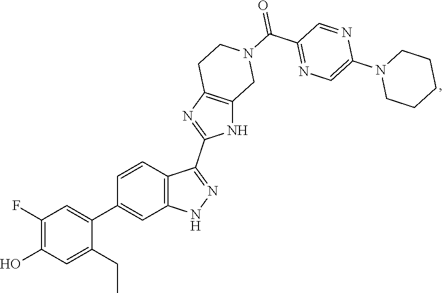 [SMILES: CCc1cc(O)c(F)cc1-c1ccc2c(-c3nc4c([nH]3)CN(C(=O)c3cnc(N5CCCCC5)cn3)CC4)n[nH]c2c1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is fedratinib (N-(tert-butyl)-3-((5-methyl-2-((4-(2-(pyrrolidin-1-yl)ethoxy)phenyl)amino)pyrimidin-4-yl)amino)benzenesulfonamide) having the following structural formula

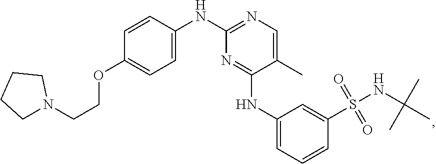 [SMILES: Cc1cnc(Nc2ccc(OCCN3CCCC3)cc2)nc1Nc1cccc(S(=O)(=O)NC(C)(C)C)c1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is cerdulatinib (4-(cyclopropylamino)-2-((4-(4-(ethyl sulfonyl)piperazin-1-yl)phenyl)amino)pyrimidine-5-carboxamide) having the following structural formula

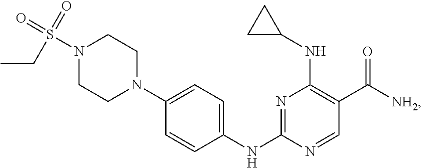 [SMILES: CCS(=O)(=O)N1CCN(c2ccc(Nc3ncc(C(N)=O)c(NC4CC4)n3)cc2)CC1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is oclacitinib (N-methyl-1-((1R,4R)-4-(methyl(7H-pyrrolo[2,3-d]pyrimidin-4-yl)amino)cyclohexyl)methanesulfonamide; CAS No. 1208319-26-9) having the following structural formula

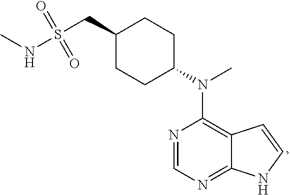 [SMILES: CNS(=O)(=O)C[C@H]1CC[C@H](N(C)c2ncnc3[nH]ccc23)CC1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is filgotinib (N-(5-(4-((1,1-dioxidothiomorpholino)methyl)phenyl)-[1,2,4]triazolo[1,5-a]pyridin-2-yl)cyclopropanecarboxamide; CAS No. 1206161-97-8) having the following structural formula

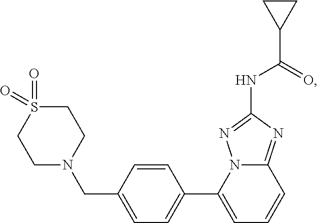 [SMILES: O=C(Nc1nc2cccc(-c3ccc(CN4CCS(=O)(=O)CC4)cc3)n2n1)C1CC1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is gandotinib (3-(4-Chloro-2-fluorobenzyl)-2-methyl -N-(5-methyl-1H-pyrazol-3-yl)-8-(morpholinomethyl)imidazo[1,2-b]pyridazin-6-amine; CAS No. 1229236-86-5) having the following structural formula

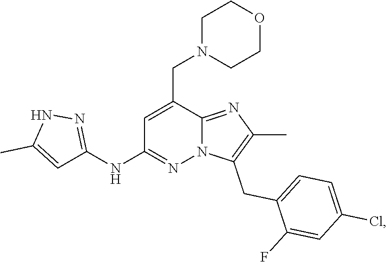 [SMILES: Cc1cc(Nc2cc(CN3CCOCC3)c3nc(C)c(Cc4ccc(Cl)cc4F)n3n2)n[nH]1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is momelotinib (N-(cyanomethyl)-4-{2-[4-(morpholin-4-yl)anilino]pyrimidin-4-yl}benzamide; CAS No. 1056637-68-4) having the following structural formula

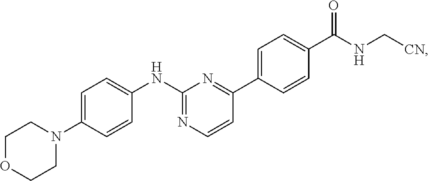 [SMILES: N#CCNC(=O)c1ccc(-c2ccnc(Nc3ccc(N4CCOCC4)cc3)n2)cc1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is pacritinib ((E)-44-(2-(pyrrolidin-1-yl)ethoxy)-6,11-dioxa-3-aza-2(4,2)-pyrimidina-1,4(1,3)-dibenzenacyclododecaphan-8-ene; CAS No. 937272-79-2) having the following structural formula

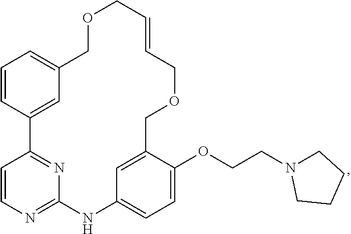 [SMILES: C1=C/COCc2cc(ccc2OCCN2CCCC2)Nc2nccc(n2)-c2cccc(c2)COC/1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is upadacitinib ((3S,4R)-3-ethyl-4-(3H-imidazo[1,2-a]pyrrolo[2,3-e]pyrazin-8-yl)-N-(2,2,2-trifluoroethyl)pyrrolidine-1-carboxamide; CAS No. 1310726-60-3) having the following structural formula

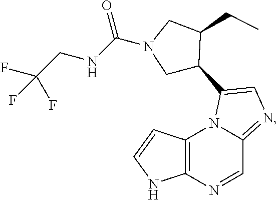 [SMILES: CC[C@@H]1CN(C(=O)NCC(F)(F)F)C[C@@H]1c1cnc2cnc3[nH]ccc3n12]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is peficitinib (4-(((1R,3S)-5-hydroxyadamantan-2-yl)amino)-1H-pyrrolo[2,3-b]pyridine-5-carboxamide; CAS No. 944118-01-8) having the following structural formula

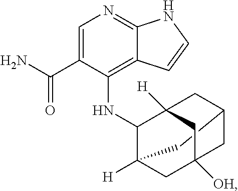 [SMILES: NC(=O)c1cnc2[nH]ccc2c1NC1[C@@H]2CC3C[C@H]1CC(O)(C3)C2]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is PF-04965842 (N-((1S,3S)-3-(methyl(7H-pyrrolo[2,3-d]pyrimidin-4-yl)amino)cyclobutyl)propane-1-sulfonamide; CAS No. 1622902-68-4) having the following structural formula

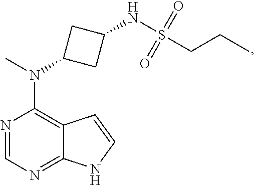 [SMILES: CCCS(=O)(=O)N[C@H]1C[C@@H](N(C)c2ncnc3[nH]ccc23)C1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is N-(3-acetamido-5-(quinoxalin-2-yl)phenyl)acrylamide having the following structural formula

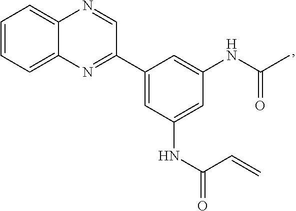 [SMILES: C=CC(=O)Nc1cc(NC(C)=O)cc(-c2cnc3ccccc3n2)c1]

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is a covalent JAK3 inhibitor. In some embodiments, the JAK/STAT modulating compound is 1-[(2S,5R)-2-Methyl-5-(7H-pyrrolo[2,3-d]pyrimidin-4-ylamino)-1-piperidinyl]-2-propen-1-one malonate (PF-06651600) having the following structural formula:

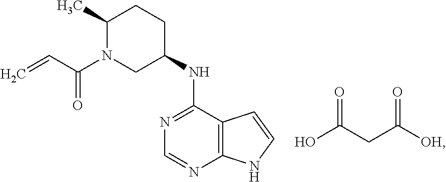

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound is [(1S)-2,2-Difluorocyclopropyl][3-[2-[(1-methyl-1H-pyrazol-4-yl)amino]-4-pyrimidinyl]-3,8-diazabicyclo[3.2.1]oct-8-yl]-methanone tosylate salt having the following structural formula:

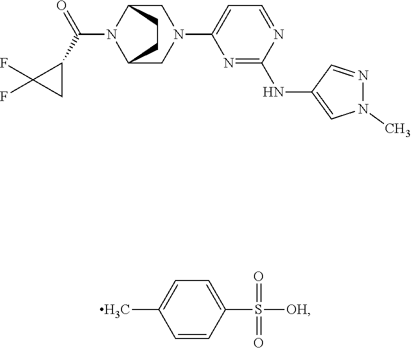

a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, or a combination thereof.

In some embodiments, the JAK/STAT modulating compound may be each administered parenterally, subcutaneously, intravenously, intramuscularly, intraperitoneally, topically, transdermally, orally, buccally, through ocular routes, intravaginally, by inhalation, by depot injections, or by implants. In some embodiments, the JAK/STAT modulating compound may be administered in an oral dosage form, such as tablets, capsules, cachets, pellets, pills, powders and granules; topical dosage forms which include, but are not limited to, a spray, extended release form, delayed release form, coated form, enteric coated form, elixir, suppository, liniment, lotion, shampoo, pastille, patch, pellet, pill, solution, powder, fluid emulsion, suspension, nanoparticle, nanoparticle suspension, nanocapsule, liposomes, nanosuspension, fluid suspension, semi-solid, ointment, paste, cream, gel, jelly, or foam.

The JAK/STAT modulating compounds of embodiments herein may also refer to a salt, solvate, N-oxide, stereoisomers, deuterated derivatives or other derivatives of the compounds of embodiments herein.

In certain embodiments, the JAK/STAT modulating compound of the present disclosure can be formulated as pharmaceutical compositions by admixture with a pharmaceutically acceptable carrier or excipient. In certain embodiments, the pharmaceutical compositions can include a therapeutically effective amount of a JAK/STAT modulating compound and a physiologically acceptable diluent or carrier. In certain embodiments, the pharmaceutical composition can further include one or more additional therapeutic components and/or adjuvants.

Compounds described herein may contain an asymmetric center and may thus exist as enantiomers. Where the compounds according to embodiments herein possess two or more asymmetric centers, they may additionally exist as diastereomers. Embodiments herein includes all such possible stereoisomers as substantially pure resolved enantiomers, racemic mixtures thereof, as well as mixtures of diastereomers. The formulas are shown without a definitive stereochemistry at certain positions. Embodiments herein includes all stereoisomers of such formulas and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general formula may be obtained by stereospecific synthesis using optically pure starting materials or reagents of known configuration. The scope of embodiments herein as described and claimed encompasses the racemic forms of the compounds as well as the individual enantiomers, diastereomers, and stereoisomer-enriched mixtures.

Conventional techniques for the preparation/isolation of individual enantiomers include chiral synthesis from a suitable optically pure precursor or resolution of the racemate using, for example, chiral high pressure liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomic mixture may be separated by chromatography and/or fractional crystallization and one or both of the diastereoisomers converted to the corresponding pure enantiomer(s) by means well known to one skilled in the art. Chiral compounds of embodiments herein (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture. Stereoisomer conglomerates may be separated by conventional techniques known to those skilled in the art. See, e.g. “Stereochemistry of Organic Compounds” by Ernest L. Eliel (Wiley, N.Y., 1994).

Atropisomers are stereoisomers resulting from hindered rotation about single bonds where the steric strain barrier to rotation is high enough to allow for the isolation of the conformers. Oki (Oki, M; Topics in Stereochemistry 1983, 1) defined atropisomers as conformers that interconvert with a half-life of more than 1000 seconds at a given temperature. The scope of embodiments herein as described and claimed encompasses the racemic forms of the compounds as well as the individual atropisomers (an atropisomer “substantially free” of tis corresponding enantionmer) and stereoisomer-enriched mixtures, i.e. mixtures of atropisomers.

Separation of atropisomers is possibly by chiral resolution methods such as selective crystallization. In an atropo-enantioselective or atroposelective synthesis one atropisomer is formed at the expense of the other. Atroposelective synthesis may be carried out by use of chiral auxiliaries like a Corey-Bakshi-Shibata (CBS) catalyst (asymmetric catalyst derived from proline) in the total synthesis of knipholone or by approaches based on thermodynamic equilibration when an isomerization reaction favors one atropisomer over the other.

As used herein, the term “a derivative thereof” refers to a salt thereof, a pharmaceutically acceptable salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, a geometric isomer thereof, a tautomer thereof, a mixture of tautomers thereof, an enantiomer thereof, a diastereomer thereof, a racemate thereof, a mixture of stereoisomers thereof, an isotope thereof (e.g., tritium, deuterium), or a combination thereof

As used herein, the term “pharmaceutically acceptable salt” refers to a salt prepared from a base or acid which is acceptable for administration to a patient, such as a mammal. The term “pharmaceutically acceptable salts” embraces salts commonly used to form alkali metal salts and to form addition salts of free acids or free bases. The nature of the salt is not critical, provided that it is pharmaceutically-acceptable. Such salts can be derived from pharmaceutically-acceptable inorganic or organic bases and from pharmaceutically-acceptable inorganic or organic acids.

Suitable pharmaceutically acceptable acid addition salts of the compounds of embodiments herein may be prepared from an inorganic acid or an organic acid. All of these salts may be prepared by conventional means from the corresponding compound of embodiments herein by treating, for example, the compound with the appropriate acid or base.

Pharmaceutically acceptable acids include both inorganic acids, for example hydrochloric, hydrobromic, hydroiodic, nitric, carbonic, sulfuric, phosphoric and diphosphoric acid; and organic acids, for example formic, acetic, trifluoroacetic, propionic, succinic, glycolic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, galacturonic, citric, fumaric, gluconic, glutamic, lactic, maleic, malic, mandelic, mucic, ascorbic, oxalic, pantothenic, succinic, tartaric, benzoic, acetic, xinafoic (1-hydroxy-2-naphthoic acid), napadisilic (1,5-naphthalenedisulfonic acid) and the like.

In some embodiments, the salt is a hydrochloride salt. In some embodiments, the salt is besylate salt. In some embodiments, the salt is a trifluoroacetate salt.

Salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including alkyl amines, arylalkyl amines, heterocyclyl amines, cyclic amines, naturally-occurring amines and the like, such as arginine, betaine, caffeine, choline, chloroprocaine, diethanolamine, N-methylglucamine, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrab amine, isopropylamine, lysine, methylglucamine, morpholine, piperazine, piperidine, polyamine resins, procaine, purines, theobromine, triethylamine, trimethylamine, tripropylamine, tromethamine and the like.

Other preferred salts according to embodiments herein are quaternary ammonium compounds wherein an equivalent of an anion (X−) is associated with the positive charge of the N atom. X− may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methanesulphonate and p-toluenesulphonate. X− is preferably an anion selected from chloride, bromide, iodide, sulphate, nitrate, acetate, maleate, oxalate, succinate or trifluoroacetate. More preferably X− is chloride, bromide, trifluoroacetate or methanesulphonate.

As used herein, an N-oxide is formed from the tertiary basic amines or imines present in the molecule, using a convenient oxidizing agent.

The compounds of embodiments herein may exist in both unsolvated and solvated forms. The term solvate is used herein to describe a molecular complex comprising a compound of embodiments herein and an amount of one or more pharmaceutically acceptable solvent molecules. The term hydrate is employed when said solvent is water. Examples of solvate forms include, but are not limited to, compounds of embodiments herein in association with water, acetone, dichloromethane, 2-propanol, ethanol, methanol, dimethylsulfoxide (DMSO), ethyl acetate, acetic acid, ethanolamine, or mixtures thereof. It is specifically contemplated that in embodiments herein one solvent molecule can be associated with one molecule of the compounds of embodiments herein, such as a hydrate.

Furthermore, it is specifically contemplated that in embodiments herein, more than one solvent molecule may be associated with one molecule of the compounds of embodiments herein, such as a dihydrate. Additionally, it is specifically contemplated that in embodiments herein less than one solvent molecule may be associated with one molecule of the compounds of embodiments herein, such as a hemihydrate. Furthermore, solvates of embodiments herein are contemplated as solvates of compounds of embodiments herein that retain the biological effectiveness of the non-solvate form of the compounds.

Embodiments herein also includes isotopically-labeled compounds of embodiments herein, wherein one or more atoms is replaced by an atom having the same atomic number, but an atomic mass or mass number different from the atomic mass or mass number usually found in nature. Examples of isotopes suitable for inclusion in the compounds of embodiments herein include isotopes of hydrogen, such as ²H and ³H, carbon, such as ¹¹C, ¹³C and ¹⁴C, chlorine, such as ³¹Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Certain isotopically-labeled compounds of embodiments herein, for example, those incorporating a radioactive isotope, are useful in drug and/or substrate tissue distribution studies. The radioactive isotopes tritium, ³H, and carbon-14, ¹⁴C, are particularly useful for this purpose in view of their ease of incorporation and ready means of detection. Substitution with heavier isotopes such as deuterium, ²H, may afford certain therapeutic advantages resulting from greater metabolic stability, for example, increased in vivo half-life or reduced dosage requirements, and hence may be preferred in some circumstances. Substitution with positron emitting isotopes, such as ₁₁C, ¹⁸F, ¹⁵O, and ¹³N, can be useful in Positron Emission Topography (PET) studies for examining substrate receptor occupancy.

Isotopically-labeled compounds of embodiments herein can generally be prepared by conventional techniques known to those skilled in the art or by processes analogous to those described herein, using an appropriate isotopically-labeled reagent in place of the non-labeled reagent otherwise employed.

Preferred isotopically-labeled compounds include deuterated derivatives of the compounds of embodiments herein. As used herein, the term deuterated derivative embraces compounds of embodiments herein where in a particular position at least one hydrogen atom is replaced by deuterium. Deuterium (D or 2H) is a stable isotope of hydrogen which is present at a natural abundance of 0.015 molar %.

Hydrogen deuterium exchange (deuterium incorporation) is a chemical reaction in which a covalently bonded hydrogen atom is replaced by a deuterium atom. Said exchange (incorporation) reaction can be total or partial.

Typically, a deuterated derivative of a compound of embodiments herein has an isotopic enrichment factor (ratio between the isotopic abundance and the natural abundance of that isotope, i.e. the percentage of incorporation of deuterium at a given position in a molecule in the place of hydrogen) for each deuterium present at a site designated as a potential site of deuteration on the compound of at least 3500 (52.5% deuterium incorporation).

In some embodiments, the isotopic enrichment factor is at least 5000 (75% deuterium). In some embodiments, the isotopic enrichment factor is at least 6333.3 (95% deuterium incorporation). In some embodiments, the isotopic enrichment factor is at least 6633.3 (99.5% deuterium incorporation). It is understood that the isotopic enrichment factor of each deuterium present at a site designated as a site of deuteration is independent from the other deuteration sites.

The isotopic enrichment factor can be determined using conventional analytical methods known to one of ordinary skilled in the art, including mass spectrometry (MS) and nuclear magnetic resonance (NMR).

Prodrugs of the compounds described herein are also within the scope of embodiments herein. Thus certain derivatives of the compounds of embodiments herein, which derivatives may have little or no pharmacological activity themselves, when administered into or onto the body may be converted into compounds of embodiments herein having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with embodiments herein can, for example, be produced by replacing appropriate functionalities present in the compounds of embodiments herein with certain moieties known to those skilled in the art as ‘pro-moieties’ as described, for example, in Design of Prodrugs by H. Bundgaard (Elsevier, 1985).

In the case of compounds of embodiments herein that are solids, it is understood by those skilled in the art that the inventive compounds and salts may exist in different crystalline or polymorphic forms, or in an amorphous form, all of which are intended to be within the scope of embodiments herein.

Some embodiments herein are directed to a pharmaceutical composition comprising a compound of embodiments herein and a pharmaceutically acceptable carrier or diluent. In some embodiments, a method of treating vitiligo comprises administering an effective amount of one or more pharmaceutical compositions comprising a JAK/STAT modulating compound of embodiments herein.

In some embodiments, the pharmaceutical compositions for use in accordance with embodiments herein can be formulated in conventional manner using one or more physiologically acceptable carriers or excipients. The therapeutic compositions of embodiments herein can be formulated for a variety of routes of administration, including systemic, topical, oral, or localized administration. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds of embodiments herein (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the compounds of embodiments herein and a suitable carrier can be solid dosage forms which include, but are not limited to, tablets, capsules, cachets, pellets, pills, powders and granules; topical dosage forms which include, but are not limited to, a solution, powder, fluid emulsion, fluid suspension, semi-solid, ointment, paste, cream, gel, jelly, or foam; and parenteral dosage forms which include, but are not limited to, solutions, suspensions, emulsions, and dry powder comprising an effective amount of a polymer or copolymer of embodiments herein. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Banker, G. S., & Rhodes, C. T. (2002). Modern pharmaceutics. New York: Marcel Dekker.; and Goodman, L. S., Brunton, L. L., Chabner, B., & Knollmann, B. C. (2011). Goodman & Gilman's pharmacological basis of therapeutics. New York: McGraw-Hill. can be consulted.

The compounds of embodiments herein can be formulated for parenteral administration by injection, e.g., by bolus injection or continuous infusion. The compounds can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as nanosuspensions, suspensions, solutions or emulsions in oily or aqueous vehicles, and can contain formulatory agents such as suspending, stabilizing and/or dispersing agents.

For administration by inhalation, the compounds for use according to embodiments herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds of embodiments herein can also be formulated in rectal compositions such as suppositories or retention enemas, e.g., containing conventional suppository bases such as cocoa butter or other glycerides.

In addition to the formulations described previously, the compounds of embodiments herein can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

For oral administration, the compounds can be formulated readily by combining the JAK/STAT modulating compounds with pharmaceutically acceptable carriers well known in the art. Such carriers enable the compounds of embodiments herein to be formulated as nanoparticles, nanoparticle suspension, tablets, troches, pills, dragees, capsules, powders, liquids, gels, syrups, slurries, suspensions and the like, for oral ingestion by a patient to be treated. Oral compositions can also be prepared using a fluid carrier for use as a mouthwash, wherein the compound in the fluid carrier is applied orally and swished and expectorated or swallowed. Pharmaceutical preparations for oral administration use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in an admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

In some embodiments, for oral administration, the pharmaceutical compositions may take the form of, for example, lozenges, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulfate). The tablets may be coated by methods well known in the art with, for example, sugars, films or enteric coatings. Additionally, the pharmaceutical compositions containing a compound of embodiments herein as active ingredient or prodrug thereof in a form suitable for oral use, may also include, for example, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient (including prodrug) in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents (for example, corn starch, or alginic acid); binding agents (for example starch, gelatin or acacia); and lubricating agents (for example magnesium stearate, stearic acid or talc). The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release. The pharmaceutical compositions described herein may also be in the form of oil-in-water emulsions.

In some embodiments, liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol, Cremophore® or fractionated vegetable oils); and preservatives (for example, methyl or propyl-p-hydroxybenzoates or sorbic acid). The preparations may also contain buffer salts, preservatives, flavoring, coloring and sweetening agents as appropriate.

In some embodiments, preparations for oral administration may be suitably formulated to give controlled release of the active compound or prodrug, as is well known.

In some embodiments, the compound is administered in a topical formulation directly to depigmented skin, or localized areas including depigmented patches (e.g., the hands or face), without applying it to any substantial amount of unaffected skin. Some embodiments are directed to a topical pharmaceutical formulation comprising a JAK/STAT modulating compound where the formulation is selected from a solution, powder, fluid emulsion, fluid suspension, semi-solid, ointment, paste, cream, gel, jelly, or foam.

Solids are generally firm and non-pourable and commonly are formulated as a bar or stick, or in particulate form; solids may be opaque or transparent, and optionally may contain solvents (including water and alcohol), emulsifiers, moisturizers, emollients, fragrances, dyes/colorants, preservatives and active ingredients. Creams and lotions are often similar to one another, differing mainly in their viscosity (creams are typically thicker and more viscous than lotions); both lotions and creams may be opaque, translucent or clear and often contain emulsifiers, solvents (including water and alcohol) and viscosity adjusting agents. Lotions and creams also may optionally contain moisturizers and emollients (especially in the case of skin care products), as well as fragrances, dyes/colorants, preservatives and active ingredients. Gels/serums may be prepared with a range of viscosities, from thick (high viscosity) to thin (low viscosity) and differ principally from lotions and creams in that gels/serums are usually clear rather than opaque. Like lotions and creams, gels/serums often contain emulsifiers, solvents (including water and alcohol) and viscosity adjusters, and may also contain moisturizers and emollients, fragrances, dyes/colorants, preservatives and active ingredients. Aqueous liquids are thinner than creams, lotions or gels, and are generally transparent; liquids usually do not contain emulsifiers. Liquid topical products often contain other solvents in addition to water (including alcohol) and may also contain viscosity adjusters, moisturizers and emollients, fragrances, dyes/colorants/pigments, preservatives and active ingredients.

In transdermal administration, the compounds of embodiments herein, for example, can be applied to a plaster, or can be applied by transdermal therapeutic systems that are consequently supplied to the organism.

Some embodiments are also directed to any of the aforementioned formulations in a kit. In some embodiments, the kit may comprise ampoules, disposable syringes, capsules, vials, tubes, or the like. In some embodiments, the kit may comprise a single dose container or multiple dose containers comprising the JAK/STAT modulating compounds of embodiments herein. In some embodiments, each dose container may contain one or more unit doses. In some embodiments, the kit may include an applicator. In some embodiments, the kit may include the JAK/STAT modulating compound in a tube having an applicator tip (e.g. a “pen”). In some embodiments, the kits include all equipment needed for combination therapy (e.g. vials of the JAK/STAT modulating compound and a phototherapy device; tubes of a JAK/STAT modulating compound in an ointment formulation and tubes having a applicator tip containing a second JAK/STAT modulating compound in a solution; or tubes of a JAK/STAT modulating compound in an ointment formulation and a second JAK/STAT modulating compound in an oral formulation). In some embodiments, the kit further includes a phototherapy device. In some embodiments, the kit containing all necessary equipment for a treatment course (e.g., 7 days of treatment). For example, the formulation may be a liquid, for example a homogeneous liquid or a suspension, sold in a bottle which dispenses the formulation as drops or a liquid film (for example from an applicator tip that contacts a target area of the skin to dispense the compound substantially only on a target area of the skin to be treated). In one embodiment, the kit includes the compound in a cream or ointment, sold in a tube. In another embodiment, the compound is provided in a viscous liquid (such as carboxylmethylcellulose, hydroxypropylmethycellulose, polyethylene glycol, glycerin, polyvinyl alcohol, or oil containing drops) for rubbing into the skin. The formulations may have preservatives or be preservative-free (for example, in a single-use container).

Pharmaceutical compositions of the compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compounds of embodiments herein can also be administered in combination with other active ingredients, such as, for example, adjuvants, protease inhibitors, or other compatible drugs or compounds where such combination is seen to be desirable or advantageous in achieving the desired effects of the methods described herein. In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the JAK/STAT modulating compound of embodiments herein can be formulated and administered to reduce the symptoms associated with vitiligo by any means that produces contact of the active ingredient with the agent's site of action in the body of a subject. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

The dosage administered may be a therapeutically effective amount of the composition sufficient to result in amelioration of symptoms of vitiligo, and can vary depending upon known factors such as the pharmacodynamic characteristics of the active ingredient and its mode and route of administration; age, sex, health and weight of the recipient; nature and extent of symptoms; kind of concurrent treatment, frequency of treatment and the effect desired.

In some embodiments, the JAK/STAT modulating compounds of embodiments herein can be administered to the subject once (e.g., as a single injection or deposition). In some embodiments, the JAK/STAT modulating compound of embodiments herein is administered at least once daily, such as at least two, three or four times daily, or are applied to the skin in a sustained release format (such as an adherent dispenser, for example a patch). In some embodiments, the JAK/STAT modulating compound may be administered daily, weekly, twice weekly, every two weeks, every three weeks, monthly, as needed, or as otherwise directed by a physician. The JAK/STAT modulating compound may be administered at any interval to achieve the therapeutically desired effect, e.g. induction or maintenance of remission. In some embodiments, the JAK/STAT modulating compound may be administered to a subject once. In some embodiments, the JAK/STAT modulating compound may be administered to a subject for a period of 1, 2, 3, 4, 5, 6, 7 days, about a week, about two weeks, about three weeks, about four weeks, about five weeks, about six weeks, about two months, about three months, about four months, about five months, about six months, or a range of any two of these values. In some embodiments, treatment may be continued for at least a week, a month, a year, or as otherwise directed by a physician. In some embodiments, treatment may extend over multiple years, the duration of disease, or the lifetime of the subject. In some embodiments, the JAK/STAT modulating compound can be administered once or twice daily to a subject in need thereof for a period of from about two to about twenty-eight days, or from about seven to about ten days. The JAK/STAT modulating compounds of embodiments herein can also be administered once or twice daily to a subject for a period of 1, 2, 3, 4, 5, 6, 7, 8, 9, 10, 11, 12 times per year, or a combination thereof.

In some embodiments the oral composition described herein is administered following an overnight fast. In some embodiments, the overnight fast is at least about 6 hours, at least about 7 hours, at least about 8 hours, at least about 9 hours, or at least about 10 hours. For example, the oral composition described herein may be administered following a high-fat meal, high-calorie meal following an overnight fast of at least 10 hours.

Furthermore, the JAK/STAT modulating compounds of embodiments herein may be co-administrated with at least one or more of the presently disclosed JAK/STAT modulating compounds or another therapeutic, either concomitantly or sequentially.

In some embodiments, the subject in need thereof is determined to have or is diagnosed with vitiligo. For example, the subject displays one or more clinical and/or histopathological features of vitiligo. In another example, the subject is first determined to be at risk of developing vitiligo. For example, the subject may have a prior personal history of vitiligo, the subject may have a family history of vitiligo and be genetically at risk of developing vitiligo, and/or the subject may have a history of a disease associated with vitiligo, such as thyroid disease.

In some embodiments, administration of one or more of the presently disclosed JAK/STAT modulating compounds is effective to cause at least partial remission, such as repigmentation of existing areas and/or reduced incidence of new areas, of the depigmented skin that characterize the disease.

In some embodiments, administration of one or more of the presently disclosed JAK/STAT modulating compounds is effective to cause at least full remission, such as repigmentation of existing areas and/or reduced incidence of new areas, of the depigmented skin that characterize the disease.

In some embodiments, administration of one or more of the presently disclosed JAK/STAT modulating compounds is effective to cause at least partial prevention, such as depigmentation of new or previously treated areas that characterize the disease.

In some embodiments, administration of one or more of the presently disclosed JAK/STAT modulating compounds is effective to cause at least full prevention, such as prevention of depigmentation of new or previously treated areas that characterize the disease.

In some embodiments, administration of one or more of the presently disclosed JAK/STAT modulating compounds is effective to prevent at least partial progression, such as prevention of increased depigmentation of existing areas and/or reduced incidence of new areas of depigmented skin that characterize the disease.

In some embodiments, administration of one or more of the presently disclosed JAK/STAT modulating compounds is effective to prevent at least full progression, such as increased depigmentation of existing areas and/or reduced incidence of new areas of the depigmented skin that characterize the disease.

In some embodiments, the JAK/STAT modulating compound may be administered at a first dose to prevent progression, at a second dose to induce remission, and/or a third dose to prevent the disease and/or maintain remission of the disease. Such doses may be the same dose, a lower dose, or a higher dose. The dose may be administered more frequently, less frequently or at the same frequency. In some embodiments, the dose may be administered in combination with another therapy, a therapeutic, an adjuvant, or the like. In some embodiments, the doses may each be administered as the same or as different dosage forms. In some embodiments, the doses may be each administered parenterally, subcutaneously, intravenously, intramuscularly, intraperitoneally, transdermally, orally, buccally, through ocular routes, intravaginally, by inhalation, by depot injections, or by implants. In some embodiments, the doses may be each administered in an oral dosage form, such as tablets, capsules, cachets, pellets, pills, powders and granules; topical dosage forms which include, but are not limited to, a a spray, extended release form, delayed release form, coated form, enteric coated form, elixir, suppository, liniment, lotion, shampoo, pastille, patch, pellet, pill, solution, powder, fluid emulsion, suspension, nanoparticle, nanoparticle suspension, nanocapsule, liposomes, nanosuspension, fluid suspension, semi-solid, ointment, paste, cream, gel, jelly, or foam.

In some embodiments, the JAK/STAT modulating compound is administered at a dose from about 0.001% w/w to about 60% w/w. In some embodiments, for oral administration, the JAK/STAT modulating compound is administered at a dose from about 0.0001 μg/kg body weight to about 60,000 μg/kg body weight. In some embodiments, the JAK/STAT modulating compound is in an amount (in w/w %) of about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 25%, about 30%, about 36%, about 40%, about 45%, about 50%, about 55%, about 60%, or a range of any two of these two values, of the oral composition.

In some embodiments, the JAK/STAT modulating compound is in an amount (in w/w %) of about 0.001% to about 60%, about 0.1% to about 60%, about 0.5% to about 60%, about 1% to about 60%, about 5% to about 60%, about 7% to about 60%, about 10% to about 60%, about 15% to about 60%, about 20% to about 60%, about 25% to about 60%, about 30% to about 60%, about 35% to about 60%, about 0.1% to about 50%, about 0.5% to about 50%, about 1% to about 50%, about 5% to about 50%, about 7% to about 50%, about 10% to about 50%, about 15% to about 50%, about 20% to about 50%, about 25% to about 50%, about 30% to about 50%, about 35% to about 50%, about 0.1% to about 40%, about 0.5% to about 40%, about 1% to about 40%, about 5% to about 40%, about 7% to about 40%, about 10% to about 40%, about 15% to about 40%, about 20% to about 40%, about 25% to about 40%, about 30% to about 40%, about 35% to about 40%, about 0.1% to about 30%, about 0.5% to about 30%, about 1% to about 30%, about 5% to about 30%, about 7% to about 30%, about 10% to about 30%, about 15% to about 30%, about 20% to about 30%, about 25% to about 30%, about 0.001% to about 20%, about 0.005% to about 20%, about 0.01% to about 20%, about 0.05% to about 20%, about 0.1% to about 20%, about 0.2% to about 20%, about 0.3% to about 20%, about 0.4% to about 20%, about 0.5% to about 20%, about 0.6% to about 20%, about 0.7% to about 20%, about 0.8% to about 20%, about 0.9% to about 20%, about 1.0% to about 20%, 1.5% to about 20%, about 2.0% to about 20%, 2.5% to about 20%, about 3.0% to about 20%, about 4% to about 20%, about 5% to about 20%, about 6% to about 20%, about 7% to about 20%, about 8% to about 20%, about 9% to about 20%, about 0.001% to about 10%, about 0.005% to about 10%, about 0.01% to about 10%, about 0.05% to about 10%, about 0.1% to about 10%, about 0.2% to about 10%, about 0.3% to about 10%, about 0.4% to about 10%, about 0.5% to about 10%, about 0.6% to about 10%, about 0.7% to about 10%, about 0.8% to about 10%, about 0.9% to about 10%, about 1.0% to about 10%, 1.5% to about 10%, about 2.0% to about 10%, 2.5% to about 10%, about 3.0% to about 10%, about 4% to about 10%, about 5% to about 10%, about 6% to about 10%, about 7% to about 10%, about 8% to about 10%, about 9% to about 10%, about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2.5%, about 0.01% to about 2%, about 0.01% to about 1.5%, about 0.01% to about 1%, about 0.01% to about 0.9%, 0.01% to about 0.8%, 0.01% to about 0.7%, 0.01% to about 0.6%, 0.01% to about 0.5%, 0.01% to about 0.4%, 0.01% to about 0.3%, 0.01% to about 0.2%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2.5%, about 0.1% to about 2%, about 0.1% to about 1.5%, about 0.1% to about 1%, about 0.1% to about 0.9%, 0.1% to about 0.8%, 0.1% to about 0.7%, 0.1% to about 0.6%, 0.1% to about 0.5%, 0.1% to about 0.4%, 0.1% to about 0.3%, 0.1% to about 0.2%, about 0.5% to about 9%, about 0.5% to about 8%, about 0.5% to about .7%, about 0.5% to about 6%, about 0.5% to about 5%, about 0.5% to about 4%, about 0.5% to about 3%, about 0.5% to about 2.5%, about 0.5% to about 2%, about 0.5% to about 1.5%, about 0.5% to about 1%, about 0.5% to about 0.9%, 0.5% to about 0.8%, 0.5% to about 0.7%, 0.5% to about 0.6%, about 1% to about 9%, about 1% to about 9%, about 1% to about 8%, about 1% to about 0.7%, about 1% to about 6%, about 1% to about 5%, about 1% to about 4%, about 1% to about 3%, about 1% to about 2.5%, about 1% to about 2%, about 1% to about 1.5% of the oral composition, or a value within any of these ranges. In some embodiments, the JAK/STAT modulating compound is in an amount of about 4% w/w.

In some embodiments, the JAK/STAT modulating compound is in an amount of about 25 mg/mL to about 500 mg/mL, about 25 mg/mL to about 250 mg/mL, about 25 mg/mL to about 100 mg/mL, about 25 mg/mL to about 75 mg/mL, about 25 mg/mL to about 60 mg/mL, about 25 mg/mL to about 50 mg/mL, about 25 mg/mL to about 45 mg/mL, about 25 mg/mL to about 40 mg/mL, about 30 mg/mL to about 60 mg/mL, about 35 mg/mL to about 60 mg/mL, about 40 mg/mL to about 60 mg/mL, or a value within any of these ranges. In some embodiments, the JAK/STAT modulating compound is in an amount of about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 125 mg/mL, about 150 mg/mL, about 250 mg/mL, about 500 mg/mL, or a range between any two of these values. The amount of the JAK/STAT modulating compound may be less when measured in its free acid form (e.g. about 40 mg/mL of sodium (5-((2-((4-fluoro-3-methoxy-5-methylphenyl)amino)-5-methylpyrimidin-4-yl)amino)-2-oxobenzo[d]oxazol-3(2H)-yl)methyl phosphate would be about 33.6 mg/mL of the free acid form). Unless otherwise stated, the amount of JAK/STAT modulating compound refers to the compound as a whole, not just the free acid/base form.

In some embodiments, the JAK/STAT compound is in a tablet or capsule form. In some embodiments, the JAK/STAT modulating compound is in an amount of about 20 mg, about 25 mg, about 50 mg, about 60 mg, about 75 mg, about 100 mg, about 110 mg, about 125 mg, about 150 mg, about 175 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320mg, about 330 mg, about 340 mg, about 350 mg, about 360 mg, about 370mg, about 380 mg, about 390 mg, about 400 mg, about 410 mg, about 420mg, about 430 mg, about 440 mg, about 450 mg, about 460 mg, about 470 mg, about 480 mg, about 490 mg, about 500 mg of the oral composition, or a value within any of these ranges. In some embodiments, the JAK/STAT modulating compound is in an amount of about 200 mg. In some embodiments, the JAK/STAT modulating compound is in an amount of about 400 mg.

In some embodiments, the effective amount of the JAK/STAT modulating compound is about 0.0001 μg/kg body weight, about 0.00025 μg/kg body weight, about 0.0005 μg/kg body weight, about 0.00075 μg/kg body weight, about 0.001 μg/kg body weight, about 0.0025 μg/kg body weight, about 0.005 μg/kg body weight, about 0.0075 μg/kg body weight, about 0.01 μg/kg body weight, about 0.025 μg/kg body weight, about 0.05 μg/kg body weight, about 0.075 μg/kg body weight, about 0.1 μg/kg body weight, about 0.25 μg/kg body weight, about 0.5 μg/kg body weight, about 0.75 μg/kg body weight, about 1 μg/kg body weight, about 5 μg/kg body weight, about 10 μg/kg body weight, about 25 μg/kg body weight, about 50 μg/kg body weight, about 75 μg/kg body weight, about 100 μg/kg body weight, about 150 μg/kg body weight, about 200 μg/kg body weight, about 250 μg/kg body weight, about 300 μg/kg body weight, about 350 μg/kg body weight, about 400 μg/kg body weight, about 450 μg/kg body weight, about 500 μg/kg body weight, about 550 μg/kg body weight, about 600 μg/kg body weight, about 650 μg/kg body weight, about 700 μg/kg body weight, about 750 μg/kg body weight, about 800 μg/kg body weight, about 850 μg/kg body weight, about 900 μg/kg body weight, about 950 μg/kg body weight, about 1,000 μg/kg body weight, about 2,000 μg/kg body weight, about 3,000 μg/kg body weight, about 4,000 μg/kg body weight, about 5,000 μg/kg body weight, about 6,000 μg/kg body weight, about 7,000 μg/kg body weight, about 8,000 μg/kg body weight, about 9,500 μg/kg body weight, about 10,000 μg/kg body weight, about 15,000 μg/kg body weight, about 20,000 μg/kg body weight, about 25,000 μg/kg body weight, about 30,000 μg/kg body weight, about 35,000 μg/kg body weight, about 40,000 μg/kg body weight, about 45,000 μg/kg body weight, about 50,000 μg/kg body weight, about 55,000 μg/kg body weight, about 60,000 μg/kg body weight, or a range between any two of these values.

In some embodiments, a therapeutically effective amount of the JAK/STAT modulating compound is about 0.1 μg/kg body weight to about 60,000 μg/kg body weight, about 0.1 μg/kg body weight to about 55,000 μg/kg body weight, about 0.1 μg/kg body weight to about 50,000 μg/kg body weight, about 0.1 μg/kg body weight to about 45,000 μg/kg body weight, about 0.1 μg/kg body weight to about 40,000 μg/kg body weight, about 0.1 μg/kg body weight to about 35,000 μg/kg body weight, about 0.1 μg/kg body weight to about 30,000 μg/kg body weight, about 0.1 μg/kg body weight to about 25,000 μg/kg body weight, about 0.1 μg/kg body weight to about 20,000 μg/kg body weight, about 0.1 μg/kg body weight to about 15,000 μg/kg body weight, about 0.1 μg/kg body weight to about 10,000 μg/kg body weight, about 0.1 μg/kg body weight to about 5,000 μg/kg body weight, about 0.1 μg/kg body weight to about 1,000 μg/kg body weight, about 0.1 μg/kg body weight to about 500 μg/kg body weight, about 0.1 μg/kg body weight to about 100 μg/kg body weight, about 0.1 μg/kg body weight to about 50 μg/kg body weight, about 0.1 μg/kg body weight to about 10 μg/kg body weight, about 0.1 μg/kg body weight to about 1μg/kg body weight, about 1μg/kg body weight to about 10,000 μg/kg body weight, about 1 μg/kg body weight to about 5,000 μg/kg body weight, about 1μg/kg body weight to about 1,000 μg/kg body weight, about 1 μg/kg body weight to about 500 μg/kg body weight, about 1 μg/kg body weight to about 100 μg/kg body weight, about 1 μg/kg body weight to about 50 μg/kg body weight, about 1 μg/kg body weight to about 10 μg/kg body weight, about 10 μg/kg body weight to about 10,000 μg/kg body weight, about 10 μg/kg body weight to about 5,000 μg/kg body weight, about 10 μg/kg body weight to about 1,000 μg/kg body weight, about 10 μg/kg body weight to about 500 μg/kg body weight, about 10 μg/kg body weight to about 100 μg/kg body weight, about 10 μg/kg body weight to about 50 μg/kg body weight, about 50 μg/kg body weight to about 10,000 μg/kg body weight, about 50 μg/kg body weight to about 5,000 μg/kg body weight, about 50 μg/kg body weight to about 1,000 μg/kg body weight, about 50 μg/kg body weight to about 500 μg/kg body weight, about 50 μg/kg body weight to about 100 μg/kg body weight, about 100 μg/kg body weight to about 10,000 μg/kg body weight, about 100 μg/kg body weight to about 5,000 μg/kg body weight, about 100 μg/kg body weight to about 1,000 μg/kg body weight, about 100 μg/kg body weight to about 500 μg/kg body weight, about 500 μg/kg body weight to about 10,000 μg/kg body weight, about 500 μg/kg body weight to about 5,000 μg/kg body weight, about 500 μg/kg body weight to about 1,000 μg/kg body weight or a value within any of these ranges.

In some embodiments, the therapeutically effective amount of the administered JAK/STAT modulating compound is about 1 mg/kg body weight, about 1.5 mg/kg body weight, about 2 mg/kg body weight, about 2.5 mg/kg body weight, about 3 mg/kg body weight, about 3.5 mg/kg body weight, about 4 mg/kg body weight, about 4.5 mg/kg body weight, about 5 mg/kg body weight, about 5.5 mg/kg body weight, about 6 mg/kg body weight, about 6.5 mg/kg body weight, about 7 mg/kg body weight, about 7.5 mg/kg body weight, about 8 mg/kg body weight, about 9.5 mg/kg body weight, about 10 mg/kg body weight, about 10.5 mg/kg body weight, about 11.0 mg/kg body weight, about 11.5 mg/kg body weight, about 12 mg/kg body weight, about 12.5 mg/kg body weight, about 13 mg/kg body weight, about 13.5 mg/kg body weight, about 14 mg/kg body weight, about 14.5 mg/kg body weight, about 15 mg/kg body weight, about 15.5 mg/kg body weight, about 16 mg/kg body weight, about 16.5 mg/kg body weight, about 17 mg/kg body weight, about 17.5 mg/kg body weight, about 18 mg/kg body weight, about 19.5 mg/kg body weight, about 20 mg/kg body weight, about 21.5 mg/kg body weight, about 22 mg/kg body weight, about 22.5 mg/kg body weight, about 23 mg/kg body weight, about 23.5 mg/kg body weight, about 24 mg/kg body weight, about 24.5 mg/kg body weight, about 25 mg/kg body weight, about 25.5 mg/kg body weight, about 26 mg/kg body weight, about 26.5 mg/kg body weight, about 27 mg/kg body weight, about 27.5 mg/kg body weight, about 28 mg/kg body weight, about 29.5 mg/kg body weight, about 30 mg/kg body weight, or a value within any of these ranges.

In some embodiments, the therapeutically effective amount of the administered JAK/STAT modulating compound is in any of the following ranges: about 1 mg/kg body weight to about 60 mg/kg body weight, about 1 mg/kg body weight to about 55 mg/kg body weight, about 1 mg/kg body weight to about 50 mg/kg body weight, about 1 mg/kg body weight to about 40 mg/kg body weight, about 1 mg/kg body weight to about 30 mg/kg body weight, about 1 mg/kg body weight to about 20 mg/kg body weight, about 1 mg/kg body weight to about 10 mg/kg body weight, about 1 mg/kg body weight to about 5 mg/kg body weight, about 5 mg/kg body weight to about 60 mg/kg body weight, about 5 mg/kg body weight to about 55 mg/kg body weight, about 5 mg/kg body weight to about 50 mg/kg body weight, about 5 mg/kg body weight to about 40 mg/kg body weight, about 5 mg/kg body weight to about 30 mg/kg body weight, about 5 mg/kg body weight to about 20 mg/kg body weight, about 5 mg/kg body weight to about 10 mg/kg body weight, about 10 mg/kg body weight to about 60 mg/kg body weight, about 10 mg/kg body weight to about 55 mg/kg body weight, about 10 mg/kg body weight to about 50 mg/kg body weight, about 10 mg/kg body weight to about 40 mg/kg body weight, about 10 mg/kg body weight to about 30 mg/kg body weight, about 10 mg/kg body weight to about 20 mg/kg body weight, about 20 mg/kg body weight to about 60 mg/kg body weight, about 20 mg/kg body weight to about 55 mg/kg body weight, about 20 mg/kg body weight to about 50 mg/kg body weight, about 20 mg/kg body weight to about 40 mg/kg body weight, about 20 mg/kg body weight to about 30 mg/kg body weight, about 30 mg/kg body weight to about 60 mg/kg body weight, about 30 mg/kg body weight to about 55 mg/kg body weight, about 30 mg/kg body weight to about 50 mg/kg body weight, about 30 mg/kg body weight to about 40 mg/kg body weight, or a value within any of these ranges.

In some embodiments, the therapeutically effective amount may be the amount administered in a day. In some embodiment, the therapeutically effective amount may be the amount administered in a dose.

In some embodiments, the JAK/STAT modulating compound of embodiments herein is administered in conjunction, concomitantly or adjunctively, with one or more additional therapeutics, such as, for example, an anti-inflammatory, an antihistamine, an antibiotic, an antiviral, an emollient, an analgesic, topical or systemic corticosteroids, topical calcineurin inhibitors, such as tacrolimus or topical pimecrolimus, local or systemic phototherapy such as, with heliotherapy, natural or artificial sunlight therapy, ultraviolet light therapy with UVB, narrow-band UVB, UVA, oral or topical photochemotherapy using psoralen (e.g. 8-methoxypsoralen (8-MOP)), or psoralen plus ultraviolet A (PUVA), calcipotriene or other topical vitamin D analogs, laser therapy, such as (e.g.) with the excimer laser (308 nm) or helium-neon (HeNe) laser, systemic immunosuppressive agents (e.g. cyclosporine A, azathioprene, cyclophosphamide), topical or oral khellin with or without phototherapy with natural sunlight or UVA, topical or oral L-phenylalanine with or without phototherapy with natural sunlight or UVA or UVB, topical and oral antioxidants (e.g. vitamin E, vitamin C, alphalipoic acid, ginkgo biloba, topical catalase, superoxide dismutase, polypodium leucotomos), surgical treatments such as, skin grafting, for example, skin minigrafting, blister grafting, punch grafting, curettage grafting, split-thickness skin grafting, transplantation of autologous melanocyte suspension, camouflage such as with make-up, tattoos (permanent dermal micropigmentation) or self-tanning agents such as dihydroxyacetone (DHA) and such, other systemic agents such as tumor necrosis factor alpha (TNFa) inhibitors, antibiotics with anti-inflammatory or immunomodulatory properties such as the tetracyclines, thyroid hormone replacement medication, other hormonal therapy, such as alpha-MSH or afamelanotide, or other therapies known to have beneficial effects in the condition, or any combination thereof.

In some embodiments, the JAK/STAT modulating compound of embodiments herein is administered in conjunction, adjunctively or concomitantly, with a biologically active agent in a topical sunscreen agent.

In some embodiments, the JAK/STAT modulating compound of embodiments herein is administered in conjunction, adjunctively or concomitantly, with phototherapy, with heliotherapy, natural or artificial sunlight therapy, ultraviolet light exposure, or combinations thereof.

In some embodiments, the JAK/STAT modulating compound disclosed in embodiments herein can also be co-administered (concurrently or sequentially) with a variety of other treatments that are not applied to the skin, for example treatments that are administered systemically, such as orally or parenterally. Examples of such systemic treatments include topical or systemic corticosteroids (such as prednisone), hormonal therapy, such as alpha-MSH or afamelanotide, antibiotics (such as erythromycin, tetracycline, and dicloxacillin), antifungal agents (such as ketoconazole and fluconazole sold under the tradename Diflucan®), antiviral agents (such as valacyclovir sold under the tradename Valtrex®, acyclovir, and famciclovir sold under the tradename Famvir®), corticosteroids, immunosuppressants (such as cyclophosphamide sold under the tradename Cytoxan®, azathioprine, methotrexate, mycophenolate), biologics (such as rituximab sold under the tradename Rituxan®, etanercept sold under the tradename Enbrel®, adalimumab sold under the tradename Humira®, infliximab sold under the tradename Remicade®, ustenkinumab sold under the tradename Stelara®, and alefacept sold under the tradename Amevive®), and/or thyroid hormone replacement, or other hormonal therapy, such as alpha-MSH or afamelanotide.

In some embodiments, other therapies that can be used in combination with the JAK/STAT modulating compounds include, for example, mercaptopurine, topical or systemic corticosteroids such as prednisone, methylprednisolone and prednisolone, alkylating agents such as cyclophosphamide, calcineurin inhibitors such as cyclosporine, sirolimus and tacrolimus, inhibitors of inosine monophosphate dehydrogenase (IMPDH) such as mycophenolate, mycophenolate mofetil, azathioprine, various antibodies, for example, antilymphocyte globulin (ALG), antithymocyte globulin (ATG), monoclonal anti-T-cell antibodies (OKT3), and irradiation. These various agents can be used in accordance with their standard or common dosages, as specified in the prescribing information accompanying commercially available forms of the drugs (see also, the prescribing information in the 2018 Edition of The Physician's Desk Reference (or current edition)). In some embodiments, standard dosages of these agents may be reduced when used in combination with one of the recited JAK/STAT modulating compounds of embodiments herein. Without limiting the scope of this disclosure, it is believed the such combination may result in synergistic results with better efficacy, less toxicity, longer duration of action, or quicker response to therapy. Azathioprine is currently available from Salix Pharmaceuticals, Inc. under the brand name Azasan®; mercaptopurine is currently available from Gate Pharmaceuticals, Inc. under the brand name Purinethol®; prednisone and prednisolone are currently available from Roxane Laboratories, Inc.; methyl prednisolone is currently available from Pfizer; sirolimus (rapamycin) is currently available from Wyeth-Ayerst under the brand name Rapamune®; tacrolimus is currently available from Fujisawa under the brand name Prograf®; cyclosporine is current available from Novartis under the brand name Sandimmune® and Abbott under the brand name Gengraf®; IMPDH inhibitors such as mycophenolate mofetil and mycophenolic acid are currently available from Roche under the brand name Cellcept® and Novartis under the brand name Myfortic®; azathioprine is currently available from Glaxo Smith Kline under the brand name Imuran®; and antibodies are currently available from Ortho Biotech under the brand name Orthoclone®, Novartis under the brand name Simulect® (basiliximab) and Roche under the brand name Zenapax® (daclizumab).

In some embodiments, the JAK/STAT modulating compound is administered either in combination or adjunctively with generalized, topical, local or systemic phototherapy (e.g., heliotherapy, natural or artificial sunlight therapy, narrow-band UV-B phototherapy, 310-315 nm), psoralen photochemotherapy (PUVA: psoralens (e.g., 5-methoxypsoralen, 8-methoxypsoralen (0.1-0.3%), trimethylpsoralen) combined with UV-A light), and/or excimer laser (308 nm) therapy. In some examples, topical tacrolimus (0.03-0.1%) ointment is combined with excimer laser therapy. Pimecrolimus (1%) cream may be combined with narrow-band UV-B treatment for vitiligo of the face. Vitamin D analogs (e.g., calcipotriol, tacalcitol) may be combined with narrow-band UV-B or PUVA treatment. In some embodiments, the compounds of embodiments herein may be administered in combination with hormonal therapies, such as alpha-MSF or afamelanotide. In some embodiments, a hormonal therapies, such as alpha-MSF or afamelanotide, may be combined with any of the foregoing therapies when administered in conjunction with the oral compositions of embodiments herein.

In some embodiments, the oral composition as described herein may be co-administrated with an ITK modulating compound, either concomitantly or sequentially. ITK inhibitors are non-receptor tyrosine kinase inhibitors of the activity of IL-2-inducible T-cell kinase (ITK), thereby interfering with the development and effector function of immune system T-cells. ITK is a key signaling component of all T-cell receptors (“TCRs”) and is also key for regulating IL-17 expression. The combination of inhibiting TCRs (inhibiting T-cell maturation and activation) as well as IL-17 means an ITK inhibitor can be thought of as a “small molecule anti-IL-17”, but with broader immunomodulatory activity. ITK inhibitors have potential therapeutic applications in autoimmune and inflammatory diseases, such as, without limitation, psoriasis and atopic dermatitis. In some embodiments, the ITK inhibitor may be selected from 1-[(3R)-3-[4-amino-3-(4-phenoxyphenyl)pyrazolo[3,4-d]pyrimidin-1-yl]piperidin-1-yl]prop-2-en-1-one (ibrutinib), 1-((S)-3-((4-((((S)-3,3-dimethylbutan-2-yl)amino)methyl)-6-(thiazolo[5,4-b]pyridin-2-ylamino)pyridin-2-yl)amino)pyrrolidin-1-yl)prop-2-en-1-one, 7-benzyl-1-(3-(piperidin-1-yl)propyl)-2-(4-(pyridin-4-yl)phenyl)-1,5-dihydro-6H-imidazo[4,5-g]quinoxalin-6-one, N-((E)-1-(((R)-1-acryloylpyrrolidin-2-yl)methyl)-5-((((S)-3,3-dimethylbutan-2-yl)amino)methyl)-1,3-dihydro-2H-benzo[d]imidazol-2-ylidene)-5-(difluoromethyl)thiophene-2-carboxamide, N-(1-((R)—((R)-1,1-dioxidotetrahydro-2H-thiopyran-2-yl)(phenyl)methyl)-1H-pyrazol-4-yl)-5,5-difluoro-5a-methyl-1,4,4a,5,5 a,6-hexahydrocyclopropa[f]indazole-3-carboxamide, (R,E)-N-(5-(((3,3-dimethylbutan-2-yl)amino)methyl)-1-(2-hydroxy-2-methylpropyl)-1H-benzo[d]imidazol-2(3H)-ylidene)-5-(1H-pyrazol-4-yl)thiophene-2-carboxamide, N-(5-((3-(4-acetylpiperazine-1-carbonyl)-4,5-dimethylbenzyl)thio)thiazol-2-yl)-4-(((3,3-dimethylbutan-2-yl)amino)methyl)benzamide, N-(5-((5-(4-acetylpiperazine-1-carbonyl)-4-methoxy-2-methylphenyl)thio)thiazol-2-yl)-4-(((3,3-dimethylbutan-2-yl)amino)methyl)benzamide, aminothiazoles, aminobenzimidazoles, indoles, aza-indoles, aza-benzimadoles, benzothiazoles, 1H-pyrazolol[4,3-c]cinnonlin-3-ol, pyridines, sulfoximine-substituted pyrimidine, aminopyridine, diazolodiazine, triazole derivatives, N-[5-[[5-[(4-Acetyl-1-piperazinyl)carbonyl]-4-methoxy-2-methylphenyl]thio]-2-thiazolyl]-4-[[(1,2,2-trimethylpropyl)amino]methyl]benzamide (CAS No. 439575-02-7), N-[5-[5-(4-acetylpiperazine-1-carbonyl)-4-methoxy-2-methylphenyl]sulfanyl-1,3-thiazol-2-yl]-4-[(3-methylbutan-2-ylamino)methyl]benzamide (CAS No. 439574-61-5), (R)-3-(1-(1-Acryloylpiperidin-3-yl)-4-amino-1H-pyrazolo[3,4-d]pyrimidin-3-yl)-N-(3-methyl-4-(1-methylethyl))benzamide (CAS No. 1407966-77-1, PF 06465469), or a combination thereof.

In some embodiments, the JAK/STAT modulating compound is administered in conjunction, concomitantly or adjunctively, with the therapies above and/or with a therapy for another disease. For example, the compound may be combined with thyroid hormone replacement therapy, other hormonal therapy, such as alpha-MSH or afamelanotide, other therapies known to have beneficial effects in the condition, with anti-inflammatory or immunomodulatory therapies, or any combination thereof.

In some embodiments, the JAK/STAT modulating compound is administered in conjunction, concomitantly or adjunctively, with an ophthalmic formulation of a drug such as an antihistamine, an antibiotic, an anti-inflammatory, an antiviral or a glaucoma medication for treating cases of vitiligo that primarily affect the eye or skin around the eye (such as the eyelids), and may be administered to or around the eye, for example in drops or ointments (e.g. a sterile ophthalmic solution, or ophthalmic ointment) for external application to the area. When preparing these combination formulations, the JAK/STAT modulating compound may be combined with ophthalmic antibiotics (such as sulfacetamide, erythromycin, gentamicin, tobramycin, ciprofloxacin or ofloxacin); ophthalmic corticosteroids (such as prednisolone, fluorometholone or dexamethasone; ophthalmic non-steroidal anti-inflammatories (such as ibuprofen, diclofenac, ketorolac or flurbiprofen); ophthalmic antihistamines (such as livostin, patanol, cromolyn, alomide, or pheniramine); ophthalmic antiviral eye medications (such as triflurthymidine, adenine, arabinoside or idoxuridine); ophthalmic glaucoma medications (for example beta-blockers such as timolol, metipranolol, carteolol, betaxolol or levobunolol); ophthalmic prostaglandin analogues (such as latanoprost); ophthalmic cholinergic agonists (such as pilocarpine or carbachol); ophthalmic alpha agonists such as bromonidine or iopidine; ophthalmic carbonic anhydrase inhibitors (such as dorzolamide); and ophthalmic adenergic agonists (such as epinephrine or dipivefrin).

In some embodiments, the JAK/STAT modulating compound is administered in conjunction, concomitantly or adjunctively, with an additional JAK/STAT modulating compound. In one embodiment, the additional JAK/STAT modulating compound can be administered topically, orally, or a combination thereof. In some embodiments, the additional JAK/STAT modulating compound may be each administered parenterally, subcutaneously, intravenously, intramuscularly, intraperitoneally, topically, transdermally, orally, buccally, through ocular routes, intravaginally, by inhalation, by depot injections, or by implants. In some embodiments, the additional JAK/STAT modulating compound may be administered in an oral dosage form, such as tablets, capsules, cachets, pellets, pills, powders and granules; topical dosage forms which include, but are not limited to, a spray, extended release form, delayed release form, coated form, enteric coated form, elixir, suppository, liniment, lotion, shampoo, pastille, patch, pellet, pill, solution, powder, fluid emulsion, suspension, nanoparticle, nanoparticle suspension, nanocapsule, liposomes, nanosuspension, fluid suspension, semi-solid, ointment, paste, cream, gel, jelly, or foam.

In some embodiments, the additional JAK/STAT modulating compound is administered at a dose from about 0.001% w/w to about 60% w/w or about 0.01% w/w to about 50% w/w. In some embodiments, for oral administration, the additional JAK/STAT modulating compound is administered at a dose from about 0.0001 μg/kg body weight to about 60,000 μg/kg body weight.

In some embodiments, the additional JAK/STAT modulating compound is administered in an amount of about 0.0001 μg/kg body weight, about 0.00025 μg/kg body weight, about 0.0005 μg/kg body weight, about 0.00075 μg/kg body weight, about 0.001 μg/kg body weight, about 0.0025 μg/kg body weight, about 0.005 μg/kg body weight, about 0.0075 μg/kg body weight, about 0.01 μg/kg body weight, about 0.025 μg/kg body weight, about 0.05%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.5%, about 4%, about 4.5%, about 5%, about 5.5%, about 6%, about 6.5%, about 7%, about 7.5%, about 8%, about 8.5%, μg/kg body weight, about 0.075 μg/kg body weight, about 0.1 μg/kg body weight, about 0.25 μg/kg body weight, about 0.5 μg/kg body weight, about 0.75 μg/kg body weight, about 9%, 1 μg/kg body weight, about 9.5%, μg/kg body weight, about 10%, μg/kg body weight, about 11%, 20 μg/kg body weight, about 12%, 25 μg/kg body weight, about 13%, 50 μg/kg body weight, about 14%, 75 μg/kg body weight, about 15%, 100 μg/kg body weight, about 16%, 150 μg/kg body weight, about 17%, 200 μg/kg body weight, about 250 μg/kg body weight, about 300 μg/kg body weight, about 350 μg/kg body weight, about 400 μg/kg body weight, about 450 μg/kg body weight, about 500 μg/kg body weight, about 550 μg/kg body weight, about 600 μg/kg body weight, about 650 μg/kg body weight, about 700 μg/kg body weight, about 750 μg/kg body weight, about 800 μg/kg body weight, about 850 μg/kg body weight, about 900 μg/kg body weight, about 950 μg/kg body weight, about 1000 μg/kg body weight, about 2000 μg/kg body weight, about 3000 μg/kg body weight, about 4000 μg/kg body weight, about 5000 μg/kg body weight, about 6000 μg/kg body weight, about 7000 μg/kg body weight, about 8000 μg/kg body weight, about 9,500 μg/kg body weight, about 10,000 μg/kg body weight, about 15,000 μg/kg body weight, about 20,000 μg/kg body weight, about 25,000 μg/kg body weight, about 30,000 μg/kg body weight, about 35,000 μg/kg body weight, about 40,000 μg/kg body weight, about 45,000 μg/kg body weight, about 50,000 μg/kg body weight, about 55,000 μg/kg body weight, about 60,000 μg/kg body weight, or a range between any two of these values.

In some embodiments, the additional JAK/STAT modulating compound is administered in an amount of about 0.1 μg/kg body weight to about 60,000 μg/kg body weight, about 0.1 μg/kg body weight to about 55,000 μg/kg body weight, about 0.1 μg/kg body weight to about 50,000 μg/kg body weight, about 0.1 μg/kg body weight to about 45,000 μg/kg body weight, about 0.1 μg/kg body weight to about 40,000 μg/kg body weight, about 0.1 μg/kg body weight to about 35,000 μg/kg body weight, about 0.1 μg/kg body weight to about 30,000 μg/kg body weight, about 0.1 μg/kg body weight to about 25,000 μg/kg body weight, about 0.1 μg/kg body weight to about 20,000 μg/kg body weight, about 0.1 μg/kg body weight to about 15,000 μg/kg body weight, about 0.1 μg/kg body weight to about 10,000 μg/kg body weight, about 0.1 μg/kg body weight to about 5,000 μg/kg body weight, about 0.1 μg/kg body weight to about 1,000 μg/kg body weight, about 0.1 μg/kg body weight to about 500 μg/kg body weight, about 0.1 μg/kg body weight to about 100 μg/kg body weight, about 0.1 μg/kg body weight to about 50 μg/kg body weight, about 0.1 μg/kg body weight to about 10 μg/kg body weight, about 0.1 μg/kg body weight to about 1μg/kg body weight, about 1 μg/kg body weight to about 60,000 μg/kg body weight, about 1 μg/kg body weight to about 55,000 μg/kg body weight, about 1 μg/kg body weight to about 50,000 μg/kg body weight, about 1 μg/kg body weight to about 45,000 μg/kg body weight, about 1 μg/kg body weight to about 40,000 μg/kg body weight, about 1 μg/kg body weight to about 35,000 μg/kg body weight, about 1 μg/kg body weight to about 30,000 μg/kg body weight, about 1 μg/kg body weight to about 25,000 μg/kg body weight, about 1 μg/kg body weight to about 20,000 μg/kg body weight, about 1 μg/kg body weight to about 15,000 μg/kg body weight, about 1 μg/kg body weight to about 10,000 μg/kg body weight, about 1 μg/kg body weight to about 5,000 μg/kg body weight, about 1 μg/kg body weight to about 1,000 μg/kg body weight, about 1 μg/kg body weight to about 500 μg/kg body weight, about 1 μg/kg body weight to about 100 μg/kg body weight, about 1 μg/kg body weight to about 50 μg/kg body weight, about 1 μg/kg body weight to about 10 μg/kg body weight, about 10 μg/kg body weight to about 10,000 μg/kg body weight, about 10 μg/kg body weight to about 5,000 μg/kg body weight, about 10 μg/kg body weight to about 1,000 μg/kg body weight, about 10 μg/kg body weight to about 500 μg/kg body weight, about 10 μg/kg body weight to about 100 μg/kg body weight, about 10 μg/kg body weight to about 50 μg/kg body weight, about 50 μg/kg body weight to about 10,000 μg/kg body weight, about 50 μg/kg body weight to about 5,000 μg/kg body weight, about 50 μg/kg body weight to about 1,000 μg/kg body weight, about 50 μg/kg body weight to about 500 μg/kg body weight, about 50 μg/kg body weight to about 100 μg/kg body weight, about 100 μg/kg body weight to about 10,000 μg/kg body weight, about 100 μg/kg body weight to about 5,000 μg/kg body weight, about 100 μg/kg body weight to about 1,000 μg/kg body weight, about 100 μg/kg body weight to about 300 μg/kg body weight, about 100 μg/kg body weight to about 500 μg/kg body weight, about 500 μg/kg body weight to about 10,000 μg/kg body weight, about 500 μg/kg body weight to about 5,000 μg/kg body weight, about 500 μg/kg body weight to about 1,000 μg/kg body weight or a value within any of these ranges.

In some embodiments, the additional JAK/STAT modulating compound is administered in an amount of about 0.1 mg/kg body weight , about 0.2 mg/kg body weight, about 0.5 mg/kg body weight, about 1 mg/kg body weight, about 1.5 mg/kg body weight, about 2 mg/kg body weight, about 2.5 mg/kg body weight, about 3 mg/kg body weight, about 3.5 mg/kg body weight, about 4 mg/kg body weight, about 4.5 mg/kg body weight, about 5 mg/kg body weight, about 5.5 mg/kg body weight, about 6 mg/kg body weight, about 6.5 mg/kg body weight, about 7 mg/kg body weight, about 7.5 mg/kg body weight, about 8 mg/kg body weight, about 9.5 mg/kg body weight, about 10 mg/kg body weight, about 10.5 mg/kg body weight, about 11.0 mg/kg body weight, about 11.5 mg/kg body weight, about 12 mg/kg body weight, about 12.5 mg/kg body weight, about 13 mg/kg body weight, about 13.5 mg/kg body weight, about 14 mg/kg body weight, about 14.5 mg/kg body weight, about 15 mg/kg body weight, about 15.5 mg/kg body weight, about 16 mg/kg body weight, about 16.5 mg/kg body weight, about 17 mg/kg body weight, about 17.5 mg/kg body weight, about 18%, mg/kg body weight, about 19%, about 20%, or a range of any two of these two values.5 mg/kg body weight, about 20 mg/kg body weight, about 21.5 mg/kg body weight, about 22 mg/kg body weight, about 22.5 mg/kg body weight, about 23 mg/kg body weight, about 23.5 mg/kg body weight, about 24 mg/kg body weight, about 24.5 mg/kg body weight, about 25 mg/kg body weight, about 25.5 mg/kg body weight, about 26 mg/kg body weight, about 26.5 mg/kg body weight, about 27 mg/kg body weight, about 27.5 mg/kg body weight, about 28 mg/kg body weight, about 29.5 mg/kg body weight, about 30 mg/kg body weight, about 35 mg/kg body weight, about 40 mg/kg body weight, about 45 mg/kg body weight, about 50 mg/kg body weight, about 55 mg/kg body weight, about 60 mg/kg body weight, or a value within any of these ranges.

In some embodiments, the additional JAK/STAT modulating compound is administering in any of the following ranges: 0.1 mg/kg body weight to about 60 mg/kg body, 0.2 mg/kg body weight to about 60 mg/kg body, 0.5 mg/kg body weight to about 60 mg/kg body, about 1 mg/kg body weight to about 60 mg/kg body weight, about 1 mg/kg body weight to about 50 mg/kg body weight, about 1 mg/kg body weight to about 40 mg/kg body weight, about 1 mg/kg body weight to about 30 mg/kg body weight, about 1 mg/kg body weight to about 20 mg/kg body weight, about 1 mg/kg body weight to about 10 mg/kg body weight, about 1 mg/kg body weight to about 5 mg/kg body weight, about 5 mg/kg body weight to about 60 mg/kg body weight, about 5 mg/kg body weight to about 50 mg/kg body weight, about 5 mg/kg body weight to about 40 mg/kg body weight, about 5 mg/kg body weight to about 30 mg/kg body weight, about 5 mg/kg body weight to about 20 mg/kg body weight, about 5 mg/kg body weight to about 10 mg/kg body weight, about 10 mg/kg body weight to about 50 mg/kg body weight, about 10 mg/kg body weight to about 40 mg/kg body weight, about 10 mg/kg body weight to about 30 mg/kg body weight, about 10 mg/kg body weight to about 20 mg/kg body weight, about 20 mg/kg body weight to about 60 mg/kg body weight, about 20 mg/kg body weight to about 50 mg/kg body weight, about 20 mg/kg body weight to about 40 mg/kg body weight, about 20 mg/kg body weight to about 30 mg/kg body weight, about 30 mg/kg body weight to about 60 mg/kg body weight, about 30 mg/kg body weight to about 50 mg/kg body weight, about 30 mg/kg body weight to about 40 mg/kg body weight, or a value within any of these ranges.

In some embodiments, the additional JAK/STAT modulating compound is administered in an amount (in w/w %) of about 0.001%, about 0.01%, about 0.05%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 25%, about 30%, about 36%, about 40%, about 45%, about 50%, about 55%, about 60%, or a range of any two of these two values.

In some embodiments, the additional JAK/STAT modulating compound is administered in an amount (in w/w %) of about 0.001% to about 60%, 0.001% to about 50%, about 0.001% to about 40%, about 0.001% to about 30%, about 0.001% to about 20%, 0.01% to about 60%, 0.01% to about 50%, about 0.01% to about 40%, about 0.01% to about 30%, about 0.01% to about 20%, about 0.05% to about 50%, about 0.05% to about 40%, about 0.05% to about 30%, about 0.05% to about 20%, about 0.1% to about 50%, about 0.1% to about 40%, about 0.1% to about 30%, about 0.1% to about 20%, about 0.2% to about 20%, about 0.3% to about 20%, about 0.4% to about 20%, about 0.5% to about 20%, about 0.6% to about 20%, about 0.7% to about 20%, about 0.8% to about 20%, about 0.9% to about 20%, about 1.0% to about 20%, 1.5% to about 20%, about 2.0% to about 20%, 2.5% to about 20%, about 3.0% to about 20%, about 4% to about 20%, about 5% to about 20%, about 6% to about 20%, about 7% to about 20%, about 8% to about 20%, about 9% to about 20%, about 0.01% to about 10%, about 0.05% to about 10%, about 0.1% to about 10%, about 0.2% to about 10%, about 0.3% to about 10%, about 0.4% to about 10%, about 0.5% to about 10%, about 0.6% to about 10%, about 0.7% to about 10%, about 0.8% to about 10%, about 0.9% to about 10%, about 1.0% to about 10%, 1.5% to about 10%, about 2.0% to about 10%, 2.5% to about 10%, about 3.0% to about 10%, about 4% to about 10%, about 5% to about 10%, about 6% to about 10%, about 7% to about 10%, about 8% to about 10%, about 9% to about 10%., about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2.5%, about 0.1% to about 2%, about 0.1% to about 1.5%, about 0.1% to about 1%, about 0.1% to about 0.9%, 0.1% to about 0.8%, 0.1% to about 0.7%, 0.1% to about 0.6%, 0.1% to about 0.5%, 0.1% to about 0.4%, 0.1% to about 0.3%, 0.1% to about 0.2%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2.5%, about 0.5% to about 2%, about 0.5% to about 1.5%, about 0.5% to about 1%, about 0.5% to about 0.9%, 0.5% to about 0.8%, 0.5% to about 0.7%, 0.5% to about 0.6%, about 1% to about 9%, about 1% to about 9%, about 1% to about 8%, about 1% to about 7%, about 1% to about 6%, about 1% to about 5%, about 1% to about 4%, about 1% to about 3%, about 1% to about 2.5%, about 1% to about 2%, about 1% to about 1.5% of the composition, or a value within any of these ranges.

In some embodiments, the JAK/STAT modulating compound can applied topically using an applicator device. In some embodiments, the applicator device permits application of the composition to a target site on the skin while preventing or at least limiting the composition's contact with non-target site areas of the skin. In some embodiments, the applicator device may allow the composition to be applied without first applying the composition to one's fingers. In some embodiments, the applicator device may include gloves, sponges, spatulas, swabs, syringes without needles, adhesive patches, or a combination thereof. In some embodiments, use of spatulas or swabs, or the like may require the device to be inserted into a container containing the composition. In some embodiments, use of syringes or adhesive patches may be accomplished by filling the syringe or patch with the composition. The composition may be expelled from the syringes onto the person's skin and/or may be topically spread on the skin by the spatulas or swabs.

In some embodiments, the JAK/STAT modulating composition is provided in an adhesive patch. Such patches generally have an adhesive layer, which is applied to a person's skin, a depot or reservoir for holding the pharmaceutical agent, and an exterior surface that prevents leakage of the pharmaceutical from the depot. The exterior surface of a patch is typically non-adhesive. In some embodiments, the JAK/STAT modulating compound incorporated into the adhesive patch may remain stable for extended periods of time.

In some embodiments, the JAK/STAT modulating compound may be incorporated into a polymeric matrix that stabilizes it, and permits the compound to diffuse from the matrix and the patch.

In some embodiments, the JAK/STAT modulating compound may be provided in one or more wells or pockets disposed near the surface of the adhesive patch that will contact the skin. The compound may also be incorporated into the adhesive layer of the patch so that once the patch is applied to the skin the compound may diffuse on to the skin or even into or through the skin. In some embodiments, the JAK/STAT modulating compound is stored in the wells in a dried, or lyophilized state. In some embodiments, the adhesive patch may contain one or more wells or pockets to hold fluid in the adhesive patch. In some embodiments, a fluid such as a gel or creams that contains water may be applied to the skin at a target site. In some embodiments, the adhesive patch containing the dried compound may be applied to the skin where the fluid may mix with the compound and the composition may move out of the patch and may move on to the skin.

In some embodiments, the transdermal patch may include a plurality of small needles that extend through the stratum corneum, but do not extend into the dermis to rupture blood vessels.

The embodiments illustrating the method and materials used may be further understood by reference to the following non-limiting examples.

### EXAMPLE 1

### Vitiligo Animal Models

An animal model may be used to screen for treatments for vitiligo. The C57BL/6J Ler-vit/vit mouse strain has been beneficial as a vitiligo research tool (Lerner et al., J. Invest. Dermatol. 87(3):299-304 (September 1986)). This strain arose from the C57BL/6J strain. The vitiligo mouse has congenital dorsal and ventral white spots as well as replacement of pigmented hairs by white hairs. The lack of pigment is due to absence of melanocytes from the epidermis and hair follicles.

Another mouse model is the C57BL/6-mivit/mvit mouse, which has a slowly progressing retinal degeneration with unevenly pigmented retinal pigment epithelium. See, e.g., Smith et al., Invest. Ophthalmol. & Vis. Sci. 35(10):3625-3632 (September 1994).

Another animal model has been developed to induce depigmentation in mice to model vitiligo. These approaches include chemically inducing melanocyte stress, immunizing mice with melanocyte antigens plus immune adjuvants to activate endogenous immune cells, or genetically altering mice to increase the frequency of melanocyte-reactive T cells. See, e.g., Essien et al., Dermatologica Sinica. 32: 240-247 (2014), incorporated herein by reference. The approach of chemically inducing melanocyte stress involves applying monobenzone to shaved abdomens of 4-week-old mice resulting in hair depigmentation. This model may help identify the mechanisms by which monobenzone-induced stress activates the immune response in vitiligo.

Another mouse model involves immunizing mice with melanocyte antigens plus immune adjuvants to activate endogenous immune cells. This approach utilizes recombinant vaccinia viruses that express a variety of melanocyte antigens. C57B16 mice are vaccinated with the recombinant vaccinia virus that expresses human TRP1 resulting in depigmentation of the hair follicle in at least 80% of mice. Depigmentation in this model is correlated with melanocyte-specific autoantibody production, and is dependent on CD4+ T cells rather than CD8+ T cells.

Another mouse model involves genetically altering mice to increase the frequency of melanocyte-reactive T cells. The AAD+ transgenic mouse expresses an MHC I molecule with the peptide binding region of human HLA-A*021 and presents tyrosinase epitope. Tyr₃₆₉. AAD+ mice were bred with albino mice that lacked the expression of tyrosinase. A T cell clone specific for Tyr₃₆₉ was isolated from these mice. The TCR genes from the clone mice were used to generate TCR transgenic mice with CD8+ T cells recognizing Tyr₃₆₉ (i.e., FH mice). When FH mice were crossed to AAD+ mice, spontaneous depigmentation occurred prominently in the hair and weakly in the tail skin. Immunohistochemistry of the affected skin and hair follicles revealed infiltrating CD8+ and CD4+ T cells. This model is dependent on CD8+ T cells, whereas CD4+ T cells are not required and negatively regulate disease. Depigmentation depended on IFN-g and the chemokine receptors CXCR3 and CCR5, which bind the IFN-g-induced chemokines CXCL9, CXCL10, and CCL5.

Another mouse model is a transgenic mouse that develops vitiligo by using mice that express a human melanocyte-specific TCR and human HLA-A2. The human TCR used to create these mice was cloned from a human CD4+ T cell infiltrating a melanoma that had high affinity for the tyrosinase₃₆₈₋₃₇₆ peptide that presented on HLA-A2, which typically interacts with CD8+ T cells.

Another mouse model is the TrpHEL model which results in hair depigmentation that is mediated by CD4+ T cell recognition of a model antigen. Membrane-bound hen egg lysozyme (HEL) is expressed in melanocytes under the control of the TRP-2 promoter. These mice are subsequently crossed to 3A9 CD4+ TCR mice, which have T cells that recognize HEL peptide 46-61. The mice developed depigmentation of their hair beginning at 21 days of age.

An alternative animal model is the Smyth line (SL) chicken developed by Dr. J. Robert Smyth, Jr. at the University of Massachusetts, Amherst, Mass. See, e.g., Shi et al., BMC Immunol. 13:18 (April 2012); Stepicheva et al., J. Immunol. 184:83.16 (2010). A partial list of some models of vitiligo that have been established in mice and chickens is set forth in Table 2, below. One knowledgeable and skilled in the art will readily appreciate that other potential animal (e.g,. mouse) models may be employed.

TABLE 2

| Model | Animal | Mechanism | Immune | Stress | Genetics | Refs |
|---|---|---|---|---|---|---|
| Smyth | Chicken | Spontaneous | T and B cells are required | Increased ROS | Inherrited | 81 |
| | | | | in feathers | depigmentation | |
| Lerner | Mouse | Spontaneous | N/A | N/A | Point mutation | 88 |
| | | | | | in MITF | |
| Zhu | Mouse | Application of MBEH to the | CD8⁺ T cells infitrate | Stress required | N/A | [symbol] |
| | | shaved abdomen of 4-week-old mice | | to induce | | |
| | | | | depigmentation | | |
| Overwijk | Mouse | Immunication on B6 mice with | CD4⁺ T cells mediate the disease | N/I | N/A | [symbol] |
| | | recombinant vaccinia virus expressing human | | | | |
| | | TRP1 | | | | |
| Bowne | Mouse | Immunication with plasmids encoding human | CD8⁺ T cells mediate the disease | N/I | N/A | [symbol] |
| | | TRP2 on gold particles via gene gun | via a perforin-dependent mechanism | | | |
| Gregg | Mouse | PMTCR transgenic mice expressing human | CD8⁺ T cells mediate the disease and | N/I | N/A | [symbol] |
| | | HLA-A*021 with CD8⁺ T cells recognizing | infiltrate the skin, CD4⁺ infiltration is | | | |
| | | Tyr₃₆₈ | also present but not required | | | |
| Metrotra | Mouse | The TCR transgene expresses the complete | CD3⁺CD8⁺CD4⁺ T cells infiltrate | N/I | N/A | 94 |
| | | HLA-A2 molecules TCRs recognize | the skin | | | |
| | | tyrosinase₃₆₈₋₃₇₈ | | | | |
| Lambe | Mouse | CD4⁺ TCR transgene recognizes HEL which is | CD4⁺ T cells mediate the disease | N/I | N/A | 95 |
| | | expressed under the control of the TRP2 promoter | | | | |
| Muranski | Mouse | TCR transgenic mouse with CD4⁺ T cells | CD4⁺ T cells mediate the disease | N/I | N/A | 96 |
| | | that recognize TRP1 | | | | |
| Antony | Mouse | Injection of B16 melanoma cells, activated | CD8⁺ T cells mediate the disease: | N/I | N/A | 97 |
| | | PMEL T cells, virus encoding hgp100 peptide and | IFN-γ is required | | | |
| | | IL-2 | | | | |
| Harris | Mouse | Adoptive transfer of TCR transgenic CD8⁺ T cells | CD8⁺ T cells mediate depigmentation | N/I | N/A | 98 |
| | | recognizing melanocyte antigen PMEL into hosts | in an IFN-γ dependent manner | | | |
| | | that return melanocytes in the epidermis | | | | |

IFN-γ = interferon-gamma;
MBEH = monobenzyl ether of hydroquinone;
MITF = microphthalmia-associated transcription Factor;
N/A = not applicable;
N/I = not investigared;
ROS = reactive oxygen species;
TCR = T cell receptor;
TRP1 = tyrosinase-related protein 1;
TRP2 = tyrosinase-related protein 2.
*The models of vitiligo are listed with identified mechanistic contributions to disease, which include melanocyte stress, autoimmunity, and genetric factors. source: Eissen et al., animal models of vitiligo: Matching the model to the question, Dermatologica Sinica 32(2014) 240-247, 246. [symbol]
indicates data missing or illegible when filed

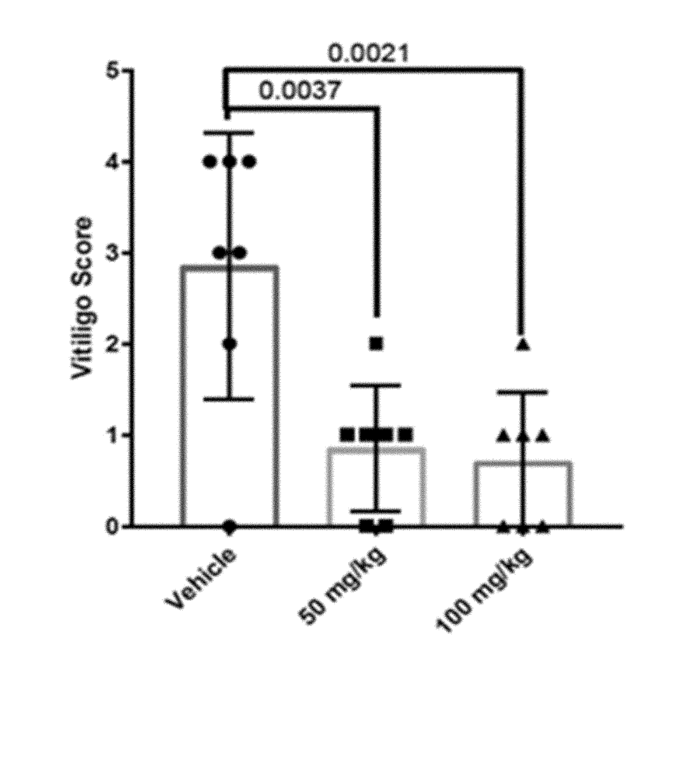

The primary driver of vitiligo is an autoimmune attack on melanocytes, the cells which give skin its color. The initiating event(s) for this autoimmune attack is unknown, however the key cell type driving the destruction of the melanocytes is the CD8+ cytolytic T cell (CTL). A major cytokine that drives the activation/function of these CTL is IFNy, whose receptor signals via JAK1 and JAK2. Drugs that block the function of JAK1 or JAK2 would be predicted to be efficacious in reversing the autoimmune destruction of melanocytes, and this has been confirmed with pilot studies using tofacitinib (an inhibitor of JAK3/JAK1/JAK2) and ruxolitinib (an inhibitor of JAK1/JAK2). The development of novel therapeutics for treatment of vitiligo would be facilitated by an animal model of disease that specifically recapitulates the key features of the disease (IFNγ CD8+ CTL driven destruction of melanocytes). Dr. John Harris of the University of Massachusetts Medical School has developed such a model. It involves the adoptive transfer of T cells from a T-cell receptor transgenic (Tg) mouse into a host mouse, and then the activation of those transgenic T cells in vivo following infection with a virus expressing its cognate antigen. This results in elevated levels of melanocytes in the epidermis and a mouse that has black hair and black skin, making the destruction of melanocytes easier to score. Attempted induction of disease is then accomplished using a proprietary method and is measured by the degree of melanocyte destruction. Common sites of melanocyte destruction (loss) are the ears, rear footpads, tail, and the nose; occasionally, the trunk skin under the hair was affected. Once the method to induce the melanocyte destruction is accomplished, the animals can be dosed with drugs to evaluate their ability to prevent disease. In ongoing work with this model, mice were orally gavaged QD with various doses of 5-((2-((4-fluoro-3-methoxy-5-methylphenyl)amino)-5-methylpyrimidin-4-yl)amino)benzo[d]oxazol-2(3H)-one hydrochloride. In-life scoring was performed by blinded investigators, and at study end, skin and lymph nodes were harvested for histology and FACS analysis of infiltrating lymphocytes. Representative in life data from a study involving animals dosed with 0, 50 or 100 mg/kg 5-((2-((4-fluoro-3-methoxy-5-methylphenyl)amino)-5-methylpyrimidin-4-yl)amino)benzo[d]oxazol-2(3H)-one hydrochloride is shown in FIG. 1. Plasma drug levels following dosing at 50 and 100 mg/kg were above the EC50 for blockade of IFNγ signaling for 6 and 20 h post dose, respectively. This is consistent with the excellent efficacy seen in this model at both doses. In FIG. 1, each symbol represents an individual animal, scored 5 weeks after initiation of dosing. Statistical significance was determined with the Student's T test.

Additional systemic (e.g., oral, gavage, intraperitoneal) and/or topical dose-ranging studies with formulations that contain a JAK/STAT modulating compound of embodiments herein are being performed in order to determine the most appropriate dosages for the investigations. Following dose selection, systemic (e.g., oral, gavage, intraperitoneal) and/or topical formulations (e.g., topical solution, foam, gel, ointment, cream, or the like) that contain a JAK/STAT modulating compound of embodiments herein will be administered by the selected systemic route or topical route to the study animal. The formulation will be administered one or more times at fixed intervals. If topically applied, the formulation will be administered to in-tact skin or following a procedure to increase penetration/permeation of the formulation such e.g., tape stripping, or followed by occlusion. The formulation may be administered once daily or multiple times each day. In some instances the formulation will be administered to the animal prior to the induction of vitiligo (a “prevention model”), in some instances the formulation will be administered at a fixed time-point after the induction and establishment of vitiligo in the animal (the “treatment model”). The animals will be observed and the therapeutic response will be assessed by measuring such clinical indicia of disease as the development of, lack of development of, or change in appearance/morphology of (e.g., the number, location, surface area or size) of pigmented and depigmented areas. Histological analysis of skin specimens will also be performed to determine the number, location, morphology of melanocytes within the skin (e.g., dermis, epidermis) and/or hair. Additional studies (such as, e.g., flow cytometry, quantitative PCR) to evaluate and quantify lymphocyte populations and subpopulations such as T-cells, CD8+ T-cells, and the like, and quantify cytokine/gene expression such as IFN-γ, CXCL9, CXCL10, CXCL11, CXCR3, CCL5, STAT1, STATS, and other cytokine levels will be performed.

Predicted Results:
1. Topically applied formulations comprising the JAK/STAT modulating compounds of embodiments herein will prevent the induction of vitiligo in the animal model. For example, it is predicted that a topical solution of 5-((2-((4-fluoro-3-methoxy-5-methylphenyl)amino)-5-methylpyrimidin-4-yl)amino)benzo[d]oxazol-2(3H)-one applied topically to a Zhu mouse before the induction of vitiligo will prevent vitiligo. As stated above, the dosage and duration of therapy will be determined through investigation before administration to the animal model.
2. Topically applied formulations comprising the JAK/STAT modulating compounds of embodiments herein will stop the progression of, improve, or cure (completely resolve) vitiligo in established disease. For example, without limitation, a topical gel comprising 5-((2-((4-fluoro-3-methoxy-5-methylphenyl)amino)-5-methylpyrimidin-4-yl)amino)benzo[d]oxazol-2(3H)-one hydrochloride applied topically to a Harris mouse with established vitiligo will improve the vitiligo. As stated above, the dosage and duration of therapy will be determined through investigation before administration to the animal model.
3. Systemically administered formulations comprising the JAK/STAT modulating compounds of embodiments herein will prevent the induction of vitiligo in the animal model. For example, without limitation, comprising 5-((2-((4-fluoro-3-methoxy-5-methylphenyl)amino)-5-methylpyrimidin-4-yl)amino)benzo[d]oxazol-2(3H)-one hydrochloride administered by injection to a Smyth chicken will prevent the induction of vitiligo. As stated above, the dosage and duration of therapy will be determined through investigation before administration to the animal model.
4. Systemically administered formulations comprising the JAK/STAT modulating compounds of embodiments herein will stop the progression of, improve, or cure (completely resolve) vitiligo in established disease. For example, without limitation, 5-((2-((4-fluoro-3-methoxy-5-methylphenyl)amino)-5-methylpyrimidin-4-yl)amino)benzo[d]oxazol-2(3H)-one administered by gavage into a Lambe mouse having established disease will completely resolve vitiligo. As stated above, the dosage and duration of therapy will be determined through investigation before administration to the animal model.
5. The topical administration of the JAK/STAT modulating compound of embodiments herein in combination with one (or more) of the known modalities to have an effect on vitiligo will prevent the induction of vitiligo in the animal model. For example, without limitation, a topical solution of disodium (5-((2-((4-fluoro-3-methoxy-5-methylphenyl)amino)-5-methylpyrimidin-4-yl)amino)-2-oxobenzo[d]oxazol-3(2H)-yl)methyl phosphate administered topically after exposure to phototherapy with natural sunlight will prevent the induction of vitiligo in Lerner mouse. As stated above, the dosage and duration of therapy will be determined through investigation before administration to the animal model.
6. The topical administration of the JAK/STAT modulating compound of embodiments herein in combination with one (or more) of the known modalities to have an effect on vitiligo will stop the progression of, improve, or cure (completely resolve) vitiligo in established disease. For example, without limitation, a topical solution comprising 5-((2-((4-fluoro-3-methoxy-5-methylphenyl)amino)-5-methylpyrimidin-4-yl)amino)benzo[d] oxazol-2(3H)-one besylate and an antioxidant administered topically will improve the appearance of vitiligo in Gregg mouse. As stated above, the dosage and duration of therapy will be determined through investigation before administration to the animal model.

The systemic administration of the JAK/STAT modulating compound of embodiments herein in combination with one (or more) of the known modalities to have an effect on vitiligo with one (or more) of the systemically administered formulations prevents the induction of vitiligo in the animal model. For example, without limitation, 5-((2-((4-fluoro-3-methoxy-5-methylphenyl)amino)-5-methylpyrimidin-4-yl)amino)benzo[d]oxazol-2(3H)-one is administered by gavage in combination with 308 nm Excimer laser therapy will prevent the induction of of vitiligo in Gregg mouse. As stated above, the dosage and duration of therapy will be determined through investigation before administration to the animal model.
8. The systemic administration of the JAK/STAT modulating compound of embodiments herein in combination with one (or more) of the known modalities to have an effect on vitiligo with one (or more) of the systemically administered formulations stops the progression of, improves, or cure (completely resolves) vitiligo in established disease. For example, without limitation, 5-((2-((4-fluoro-3-methoxy-5-methylphenyl)amino)-5-methylpyrimidin-4-yl)amino)benzo[d]oxazol-2(3H)-one hydrochloride administered by injection to a Smyth chicken in combination with psoralen plus ultraviolet A (PUVA) therapy stops the progression of vitiligo. As stated above, the dosage and duration of therapy will be determined through investigation before administration to the animal model.
9. Pretreating or concomitantly treating an animal model or a human with a modality (such as, UVA, UVB , or even natural sunlight) prior to dosing with a formulation of the invention (topical or systemic) will or may be required to show clinical efficacy or optimal clinical efficacy. For example, without limitation, a subject pretreated (e.g. 1-3 days prior) with ultraviolet light therapy with UVB before administration of a topical solution of 5-((2-((4-fluoro-3-methoxy-5-methylphenyl)amino)-5-methylpyrimidin-4-yl)amino)benzo[d]oxazol-2(3H)-one may show clinical efficacy (e.g. improvement in the appearance of the vitiligo). As stated above, the dosage and duration of therapy will be determined through investigation before administration to the subject.
10. Treatment of clinically affected skin (e.g. already depigmented patches) as well as clinically as yet unaffected skin (e.g. areas immediately surrounding or “mirror-image” (other-side-of-the-body) areas) may prevent or ameliorate the disease. For example, without limitation, a topical cream comprising 5-((2-((4-fluoro-3-methoxy-5-methylphenyl)amino)-5-methylpyrimidin-4-yl)amino)benzo[d]oxazol-2(3H)-one trifluoroacetate administered topically to depigmented skin as well as areas immediately surrounding the skin may improve the appearance of vitiligo in subjects. As stated above, the dosage and duration of therapy will be determined through investigation before administration to the animal model.

### EXAMPLE 2

### Topical Treatment Using 5-((2-((4-Fluoro-3-Methoxy-5-Methylphenyl)Amino)-5-Methylpyrimidin-4-yl)Amino)Benzo[D]Oxazol-2(3H)-One Hydrochloride

Mammalian subjects to be treated with the claimed compounds will be selected based on a clinical and/or histopathological presentation of vitiligo. A 0.46% (w/w) solution formulation of 5-((2-((4-fluoro-3-methoxy-5-methylphenyl)amino)-5-methylpyrimidin-4-yl)amino)benzo[d]oxazol-2(3H)-one hydrochloride will be provided in an ampoule. The formulation will be applied topically to the depigmented areas on the skin such as patches on the face and extremities with a applicator (such as a swab) to carefully spread the formulation over the affected areas without spreading to surrounding unaffected skin tissue. The pharmaceutical formulation is applied to the depigmented patches daily, for example 2-4 times per day for more than one day, for example, at least sixteen weeks. Topical application of the formulation to the depigmented areas is continued until the areas to which the formulation is applied show evidence of repigmentation or disappear, or their progression is delayed or stopped.

### EXAMPLE 3

### Oral Treatment using 5-((2-((4-Fluoro-3-Methoxy-5-Methylphenyl)Amino)-5-Methylpyrimidin-4-yl)Amino)Benzo[D]Oxazol-2(3H)-One Hydrochloride

A subject having white macules and patches on the forehead, trunk, and extremities will be treated with oral 5-((2-((4-fluoro-3-methoxy-5-methylphenyl)amino)-5-methylpyrimidin-4-yl)amino)benzo[d]oxazol-2(3H)-one hydrochloride at a dosage of 5 mg every other day. After 3 weeks, the dosage will be increased to 5 mg/day. It is believed that the subject will show clinically significant improvement within 6 months and that the subject will tolerate the therapeutic compound without adverse effects. The subject will also be assessed to measure complete blood cell count, serum creatinine, hepatic function, or lipids during the course of treatment. It is expected that no abnormalities will result from this treatment.

### EXAMPLE 4

### Topical Treatment using 5-((2-((4-Fluoro-3-Methoxy-5-Methylphenyl)Amino)-5-Methylpyrimidin-4-yl)Amino)Benzo[D]Oxazol-2(3H)-One Hydrochloride

Subjects with a clinical diagnosis of new onset or actively progressing non-segmental facial vitiligo or worsening of existing facial lesions within the past 6 months were enrolled in an open-label clinical trial and treated with a 0.46% (w/w) topical solution formulation of 5-((2-((4-fluoro-3-methoxy-5-methylphenyl)amino)-5-methylpyrimidin-4-yl)amino)benzo[d]oxazol-2(3H)-one hydrochloride applied twice daily for 24 weeks by the protocol-prescribed method. As early as 4 weeks after initiating treatment subjects were observed to be evidencing early improvement in their disease such as, improvement in pigmentation at the borders of the vitiliginous lesions and/or improvement in areas of depigmentation (repigmentation) as judged by clinical photographs and/or improvement in the Facial VASI index.

It is anticipated that clinically significant improvement of disease (a response to treatment) will be demonstrated in all or a subset of subjects as measured by assessment of response to treatment by clinical evaluation, photographic digital image analysis, Facial VASI (Vitiligo Activity Severity Index) Score, the Vitiligo Noticeability Scale (VNS), VitiQoL (Vitiligo quality of life index) and/or the Subject Vitiligo Satisfaction Scale during the 24 weeks of active treatment and/or the 4 week post treatment follow-up period. It is anticipated that the treatment will be judged to be safe and tolerable.

Although embodiments herein has been described in considerable detail with reference to certain preferred embodiments thereof, other versions are possible. Therefore, the spirit and scope of the appended claims should not be limited to the description and the preferred versions contained within this specification.

1. A method of treating vitiligo in a subject in need thereof comprising administering to the subject a therapeutically effective amount of a JAK/STAT modulating compound selected from the group consisting of 5-((2-((4-fluoro-3-methoxy-5-methylphenyl)amino)-5-methylpyrimidin-4-yl)amino)benzo[d]oxazol-2(3H)-one, disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, tofacitinib, ruxolitinib, baricitinib, lestaurtinib, decernotinib, TG101348, Janex 1, PF-956980, WHI-P154, ZM-39923, NSC114792, cerdulatinib, fedratinib, PF-06263276, CEP-33779, AZD-1480, SHR0302, oclacitinib, filgotinib, gandotinib, momelotinib, pacritinib, upadacitinib, peficitinib, PF-04965842, N-(3-acetamido-5-(quinaxalin-2-yl) phenyl) acrylamide, 1-[(2S,5R)-2-Methyl-5-(7H-pyrrolo[2,3-d]pyrimidin-4-ylamino)-1-piperidinyl]-2-propen-1-one malonate (PF-06651600), [(1S)-2,2-Difluorocyclopropyl][3-[2-[(1-methyl-1H-pyrazol-4-yl)amino]-4-pyrimidinyl]-3,8-diazabicyclo[3.2.1]oct-8-yl]-methanone tosylate, a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, and a combination thereof.

2. The method of claim 1, wherein the JAK/STAT modulating compound is disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, and a combination thereof.

3. The method of claim 1, wherein the JAK/STAT modulating compound is 5-((2-((4-fluoro-3-methoxy-5-methylphenyl)amino)-5-methylpyrimidin-4-yl)amino)benzo[d]oxazol-2(3H)-one, a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, and a combination thereof.

4. The method of claim 1, wherein the JAK/STAT modulating compound is administered at a dose from about 0.001% w/w to about 60% w/w.

5. The method of claim 1, wherein the JAK/STAT modulating compound is administered at a dose from about 0.0001 μg/kg body weight to about 60,000 μg/kg body weight.

6. The method of claim 1, wherein route of administration is selected from the group consisting of oral, topical, systemic, parenteral, subcutaneous, intramuscular, intraperitoneal, transdermal, intravenous, rectal, local, sublingual, buccal, ocular, intravaginal, by inhalation, by depot injection, by implant, and a combination thereof.

7. The method of claim 1, wherein the compound is administered either in combination or adjunctively with an additional JAK/STAT modulating compound or another therapeutic modality.

8. The method of claim 1, wherein the compound is administered either in combination or adjunctively with phototherapy, natural sunlight, heliotherapy, artificial sunlight therapy, ultraviolet light exposure, or combinations thereof.

9. A method of treating vitiligo in a subject in need thereof comprising topically administering to the subject a JAK/STAT modulating compound selected from 5-((2-((4-fluoro-3-methoxy-5-methylphenyl)amino)-5-methylpyrimidin-4-yl)amino)benzo[d]oxazol-2(3H)-one, disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, tofacitinib, ruxolitinib, baricitinib, lestaurtinib, decernotinib, TG101348, Janex 1, PF-956980, WHI-P154, ZM-39923, NSC114792, cerdulatinib, fedratinib, PF-06263276, CEP-33779, AZD-1480, SHR0302, oclacitinib, filgotinib, gandotinib, momelotinib, pacritinib, upadacitinib, peficitinib, PF-04965842, N-(3-acetamido-5-(quinaxalin-2-yl) phenyl) acrylamide, 1-[(2S,5R)-2-Methyl-5-(7H-pyrrolo[2,3-d]pyrimidin-4-ylamino)-1-piperidinyl]-2-propen-1-one malonate (PF-06651600), [(1S)-2,2-Difluorocyclopropyl][3-[2-[(1-methyl-1H-pyrazol-4-yl)amino]-4-pyrimidinyl]-3,8-diazabicyclo[3.2.1]oct-8-yl]-methanone tosylate, a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, and a combination thereof.

10. The method of claim 9, wherein the JAK/STAT modulating compound is 5-((2-((4-fluoro-3-methoxy-5-methylphenyl)amino)-5-methylpyrimidin-4-yl)amino)benzo[d]oxazol-2(3H)-one, a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, and a combination thereof.

11. The method of claim 9, wherein the JAK/STAT modulating compound is 5-((2-((4-fluoro-3-methoxy-5-methylphenyl)amino)-5-methylpyrimidin-4-yl)amino)benzo[d]oxazol-2(3H)-one hydrochloride.

12. The method of claim 9, wherein the JAK/STAT modulating compound is administered at a dose from about 0.001% w/w to about 60% w/w.

13. The method of claim 9, wherein the compound is administered either in combination or adjunctively with an additional JAK/STAT modulating compound or another therapeutic modality.

14. The method of claim 9, wherein the JAK/STAT modulating compound is in a spray, liniment, lotion, shampoo, conditioner, patch, solution, powder, fluid emulsion, suspension, nanoparticle, nanoparticle suspension, nanocapsule, liposomes, nanosuspension, fluid suspension, semi-solid, ointment, paste, cream, gel, jelly, or foam.

15. A method of treating vitiligo in a subject in need thereof comprising orally administering to the subject a therapeutically effect amount of a JAK/STAT modulating compound selected from 5-((2-((4-fluoro-3-methoxy-5-methylphenyl)amino)-5-methylpyrimidin-4-yl)amino)benzo[d]oxazol-2(3H)-one, disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, tofacitinib, ruxolitinib, baricitinib, lestaurtinib, decernotinib, TG101348, Janex 1, PF-956980, WHI-P154, ZM-39923, NSC114792, cerdulatinib, fedratinib, PF-06263276, CEP-33779, AZD-1480, SHR0302, oclacitinib, filgotinib, gandotinib, momelotinib, pacritinib, upadacitinib, peficitinib, PF-04965842, N-(3-acetamido-5-(quinaxalin-2-yl) phenyl) acrylamide, 1-[(2S,5R)-2-Methyl-5-(7H-pyrrolo[2,3-d]pyrimidin-4-ylamino)-1-piperidinyl]-2-propen-1-one malonate (PF-06651600), [(1S)-2,2-Difluorocyclopropyl][3-[2-[(1-methyl-1H-pyrazol-4-yl)amino]-4-pyrimidinyl]-3,8-diazabicyclo[3.2.1]oct-8-yl]-methanone tosylate, a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, and a combination thereof.

16. The method of claim 15, wherein the JAK/STAT modulating compound is 5-((2-((4-fluoro-3-methoxy-5-methylphenyl)amino)-5-methylpyrimidin-4-yl)amino)benzo[d]oxazol-2(3H)-one, a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, and a combination thereof.

17. The method of claim 15, wherein the JAK/STAT modulating compound is disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, and a combination thereof.

18. The method of claim 15, wherein the JAK/STAT modulating compound is administered at a dose from about 0.001% w/w to about 60% w/w.

19. The method of claim 15, wherein the JAK/STAT modulating compound is administered at a dose from about 0.0001 μg/kg body weight to about 60,000 μg/kg body weight.

20. The method of claim 15, wherein the compound is administered either in combination or adjunctively with an additional JAK/STAT modulating compound or another therapeutic modality.

21. The method of claim 15, wherein the JAK/STAT modulating compound is in a pharmaceutical composition further comprising a pharmaceutically acceptable excipient.
